(19) United States
(12) Patent Application Publication
(10) Pub. No.: US 2024/0074816 A1
(43) Pub. Date: Mar. 7, 2024

(54) STERILIZABLE IMAGE MANIPULATION DEVICE

(71) Applicants: Dmitry Levin, Washington, DC (US); UNITED STATES GOVERNMENT AS REPRESENTED BY THE DEPARTMENT OF VETERANS AFFAIRS, Washington, DC (US)

(72) Inventors: Dmitry Levin, Washington, DC (US); Beth Ripley, Washington, DC (US); William Nylander, Washington, DC (US)

(21) Appl. No.: 18/270,415

(22) PCT Filed: Dec. 30, 2021
(86) PCT No.: PCT/US21/65674

§ 371 Date: Jun. 29, 2023

Related U.S. Application Data
(60) Provisional application No. 63/132,164, filed on Dec. 30, 2020.

(51) Int. Cl. A61B 34/20; A61B 90/96; G06K 19/06; G06T 7/70; G06T 19/00; G16H 10/60

(52) U.S. Cl. CPC A61B 34/20; A61B 90/96; G06K 19/06028; G06K 19/06037; G06T 7/70; G06T 19/00; G16H 10/60; A61B 2090/3937; G06T 2207/30004; G06T 2210/41

(57) ABSTRACT

Methods, systems, and apparatuses are described for a sterilizable image manipulation device to be used in medical procedures. An orientation of the sterilizable image manipulation device may be determined and synchronized with an orientation of a virtual object. A change in the orientation of the sterilizable image manipulation device may be represented as a change in the orientation of the virtual object.

### CROSS-REFERENCE TO RELATED APPLICATION

This application claims the benefit of U.S. Provisional Application No. 63/132,164, filed Dec. 30, 2020, which is hereby incorporated by reference in its entirety.

### BACKGROUND

During a surgical procedure, it is often necessary for a proceduralist to refer back to pre-procedural medical imaging datasets, such as computed tomography (CT) or magnetic resonance imaging (MRI) scans, to confirm patient-specific anatomical landmarks and the location of pathology. Proceduralist refers to surgeons, interventional cardiologists, interventional radiologists, physicians, physician assistants and others who perform invasive procedures. To view a patient's medical images during a procedure, proceduralists have limited options. The first option is for the proceduralist to break the sterile field (remove surgical gown and gloves) so he or she can review images on a nearby computer. A drawback to this approach is lost time by scrubbing out and into the procedure, potential risk of infection from crossing the sterile field multiple times, and disruption to the flow of the procedure as the team pauses for the proceduralist to review images. Another option is that the proceduralist asks someone outside of the sterile field to scroll through images for them, with images viewed on a screen that the proceduralist can see without leaving the sterile field. However, drawbacks include an additional layer of communication needed for the proceduralist to direct the person manipulating images to what they need to see, as well as the loss of additional relational understanding that the proceduralist gains from manipulating the data him or herself. An additional approach may be to introduce a device such as augmented reality (AR) glasses so the proceduralist can view the images. However, the hand commands needed to manipulate images using augmented reality glasses are confusing and challenging because dynamic movements and multiple sets of hands may be involved in the surgery. Further, there is reluctance on the part of some proceduralists to adopt technology that might interfere with glasses or other headgear worn by proceduralists during procedures. Thus, improved medical imaging processing methods and systems are needed.

### SUMMARY

Methods, systems and apparatuses are provided for a sterilizable tool that a proceduralist can use during surgeries and other procedures to manipulate image data in real time. The augmented reality (AR) device may generate image data which may be observed by a user (for example a proceduralist) wearing/using the AR device. Although discussed generally herein in connection with “augmented reality,” when the words “augmented reality” are used, it should be understood that this also encompasses “virtual reality,” “mixed reality,” “extended reality,” and other experiences involving the combination of any real object with a two-dimensional or three-dimensional immersive environment or experience.

The image cube may comprise a sterilizable material which can be within the sterile field, which may allow a proceduralist to manipulate medical images (for instance, CT, MRI, fluoroscopic and other images retrieved from an electronic health record) without breaking the sterile field. The image cube may comprise a material which has sufficient contrast so as to be imaged by a camera mounted above or adjacent to the surgical field and thereby facilitate the rendering of a virtual object. The location of the image cube in space may be determined by a variety of sensors. The image cube may be in communication with a plurality of computing devices including servers, displays, image capture technologies and the like. Thus, an operating environment may comprise a surgical room whereby a camera or other image capture device is mounted above the surgical field and the feed from the camera (with the image cube in the field of view) is routed to at least a display. The system may associate an augmented reality or virtual reality image of, for example, a body part, with the image cube. Thus, a proceduralist may interact with the AR or VR image of the body part by manipulating the image cube. The system may comprise software which maintains HIPAA compliant handling of data including medical images and the like.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

To easily identify the discussion of any particular element or act, the most significant digit or digits in a reference number may refer to the figure number in which that element is first introduced.

FIG. 1 shows an example computing environment;

FIG. 2 shows an example device layout;

FIGS. 3A-3H show example device configurations;

FIG. 4 shows an example method;

FIG. 5 shows an example method;

FIG. 6 shows an example environment;

FIG. 7 shows an example environment;

FIG. 8 shows an example display;

FIG. 9 shows an example display;

FIG. 10 shows an example display;

FIG. 11 shows an example computing environment;

FIG. 12 shows an example device;

FIG. 13 shows an example environment; and

FIG. 14 shows an example method.

### DETAILED DESCRIPTION

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes—from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the examples included therein and to the Figures and their previous and following description.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

Hereinafter, various embodiments of the present disclosure will be described with reference to the accompanying drawings. As used herein, the term “user” may indicate a person who uses an electronic device or a device (e.g., an artificial intelligence electronic device) that uses an electronic device.

The present disclosure relates to a sterilizable image cube for use in medical procedures. The image cube may comprise a sterilizable material which can be within the sterile field, which may allow a proceduralist to manipulate medical images (for instance, CT, MRI, fluoroscopic and other images retrieved from an electronic health record) without breaking the sterile field. The image cube may comprise one or more features (e.g., surface features) which are distinguishable (e.g., via computer vision techniques) from each other.

The image cube may comprise a material which has sufficient contrast so as to be imaged by a camera mounted above or adjacent to the surgical field and thereby facilitate the rendering of a virtual object. The location of the image cube in space may be determined by a variety of sensors such as computer vision sensors so as to determine an orientation of the image cube and a position in space of the image cube. The image cube may be in communication with a plurality of computing devices including servers, displays, image capture technologies and the like. The system may associate an augmented reality or virtual reality image of, for example, a body part, with the image cube. For example, the orientation of the augmented or virtual reality body part may be synchronized with the orientation of the image cube may anchoring (e.g., virtually registering) features of the augmented or virtual reality heart with the features of the image cube. Thus, a proceduralist may interact with the AR or VR image of the body part by manipulating the image cube.

An AR device may comprise, or be in communication with, a camera, which may be, for example, any imaging device and/or video device such as a digital camera and/or digital video camera. Throughout the specification, reference may be made to an AR/VR/XR device. “AR” may refer to augmented reality, “XR” may refer to extended reality, and “VR” may refer to virtual reality. It is to be understand that AR, XR, and VR may be used interchangeably and refer to the same circumstances or devices etc. The AR device may utilize the camera to capture one or more images (e.g., image data) in the field of view, process the image data, and cause output of processed image data (e.g., on a display of the AR device or on a separate display). The image data may include, for example, data associated with the one or more physical objects (e.g., a person, an organ, a medical implement, a tool, or the like) in the augmented reality scene and/or virtual representations thereof, including for example, three-dimensional (3D) spatial coordinates of the one or more physical objects. The AR device may also comprise one or more sensors configured to receive and process image data and/or orientation data associated with the AR device. The orientation data may include, for example, data associated with roll, pitch, and yaw rotations. Additional sensors and/or data may be obtained, for example, LIDAR, radar, sonar, signal data (e.g., received signal strength data), and the like.

One or more of the image data, the orientation data, combinations thereof, and the like, may be used to determine AR image data associated with the augmented reality scene. For example, spatial data may be associated with the one or more physical objects in the augmented reality scene combinations thereof, and the like. The spatial data may comprise data associated with a position in 3D space (e.g., x, y, z coordinates). The position in 3D space may comprise a position defined by a center of mass of a virtual or physical object and/or a position defined by one or more boundaries (e.g., outline or edge) of the virtual or physical object.

Depending on the AR application, one or more virtual objects of varying size, shape, orientation, color, and the like may be determined. For example, in a surgical application, a virtual representation of a human heart may be determined. Likewise, in an engineering application, a virtual representation of a mechanical part may be determined. In an additional exemplary application such as an AR gaming application, a virtual sword or animal may be determined. Spatial data associated with the one or more virtual objects may be determined. The spatial data associated with the one or more virtual objects may be registered to spatial data associated with the image cube. Registering may refer to determining the position of a given virtual object of the one or more virtual objects w/in the scene. Registering may also refer to the position of the virtual object relative any of the one or more physical objects in the augmented reality scene. The system may associate an augmented reality or virtual reality image of, for example, a body part, with the image cube. Thereby, a proceduralist may interact with the AR or VR image of the body part by manipulating the image cube.

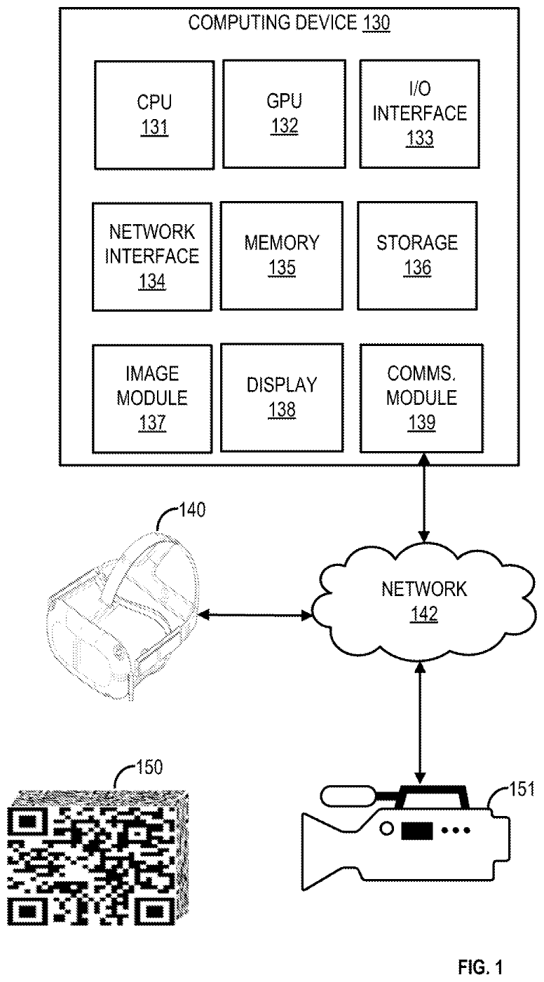

FIG. 1 shows an example system 100 for interacting with an augmented reality environment using a three-dimensional object. The system 100 includes a computing device 130, an AR device 140, a three-dimensional object 150, and an image capture device such a camera 151. The image capture device may be separate from and/or incorporated into the computing device 130 as described below. Multiple computing devices may be used, but only one is required.

The computing device 130 includes a central processing unit (CPU) 131, a graphics processing unit (GPU) 132, an input-output (I/O) interface 133, a network interface 134, memory 135, storage 136, an image module 137, and a display 138.

The CPU 131 may execute instructions associated with an operating system for the computing device 130 as well as instructions associated with one or more applications suitable for enabling the functions described herein. The CPU 131 may be or include one or more microprocessors, microcontrollers, digital signal processors, application specific integrated circuits (ASICs), or a system-on-a-chip (SOCs). The CPU 131 may be specialized, designed for operations upon visual, graphical, or audio data or may be general purpose processors. Though identified as a central processing unit, the CPU 131 may in fact be multiple processors, for example multi-core processors or a series of processors joined by a bus to increase the overall throughput or capabilities of the CPU 131. For purposes of performing the tracking described here, the CPU may be, in whole or in part, an all-in-one “motion chip” designed expressly for the purpose of enabling three-dimensional object tracking.

The GPU 132 may execute instructions suitable for enabling the functions described herein. In particular, the GPU 132 may be used in connection with particular image-related operations which the GPU 132 is uniquely suited to perform such as rendering or complex mathematical calculations related to object detection and computer vision. The GPU 132 may be any of the things that the CPU 131 is. However, the GPU 132 is distinct in that it is a specialized processor that is designed for the purpose of processing visual data, particularly vector and shading operations, performs faster memory operations and access, and is capable of performing specialized lighting operations within rendered three-dimensional environments. The instruction sets and memory in the GPU 132 are specifically designed for operation upon graphical data. In this way, the GPU 132 may be especially suited to operation upon the image data or to quickly and efficiently performing the complex mathematical operations described herein like the CPU 131, the GPU 132 is shown as a single graphics processing unit, but may actually be one or more graphics processing units in a so-called multi-core format or linked by a bus or other connection that may together be applied to a single set of or to multiple processing operations.

The I/O interface 133 may include one or more general purpose wired interfaces (e.g. a universal serial bus (USB), high definition multimedia interface (HDMI)), one or more connectors for storage devices such as hard disk drives, flash drives, or proprietary storage solutions.

The I/O interface 133 may be used to communicate with and direct the actions of optional, external sensors such as additional cameras, lights, infrared lights, or other systems used for or in the process of performing computer vision detection and other operations on the three-dimensional object 150.

The network interface 134 may include radio-frequency circuits, analog circuits, digital circuits, one or more antennas, and other hardware, firmware, and software necessary for network communications with external devices. The network interface 134 may include both wired and wireless connections. For example, the network may include a cellular telephone network interface, a wireless local area network (LAN) interface, and/or a wireless personal area network (PAN) interface. A cellular telephone network interface may use one or more cellular data protocols. A wireless LAN interface may use the WiFi® wireless communication protocol or another wireless local area network protocol. A wireless PAN interface may use a limited-range wireless communication protocol such as Bluetooth®, WiFi®, ZigBee®, or some other public or proprietary wireless personal area network protocol.

The network interface 134 may include one or more specialized processors to perform functions such as coding/decoding, compression/decompression, and encryption/decryption as necessary for communicating with external devices using selected communications protocols. The network interface 134 may rely on the CPU 131 to perform some or all of these functions in whole or in part.

The memory 135 may include a combination of volatile and/or non-volatile memory including read-only memory (ROM), static, dynamic, and/or magnetoresistive random access memory (SRAM, DRM, MRAM, respectively), and nonvolatile writable memory such as flash memory.

The memory 135 may store software programs and routines for execution by the CPU 131 or GPU 132 (or both together). These stored software programs may include operating system software. The operating system may include functions to support the I/O interface 133 or the network interface 134, such as protocol stacks, coding/decoding, compression/decompression, and encryption/decryption. The stored software programs may include an application or “app” to cause the computing device to perform portions or all of the processes and functions described herein. The words “memory” and “storage”, as used herein, explicitly exclude transitory media including propagating waveforms and transitory signals.

Storage 136 may be or include non-volatile memory such as hard disk drives, flash memory devices designed for long-term storage, writable media, and other proprietary storage media, such as media designed for long-term storage of image data.

The image module 137 may be configured to receive, process, generate, store, send or otherwise process image data. For example, the image module may comprise a camera. The image module 137 is shown as a single camera, but may be a dual- or multi-lens camera. The image module 137 may receive, for example, captured image data. For example, the image module 137 may receive, from camera 151, captured image data. The captured image data may comprise image data associated with the image cube 150. The image module 137 may process the captured image data and determine, for example, an orientation associated with the cube 150. The image module 137 may determine the orientation of the image cube via computer vision techniques. For example, the image module 137 may different one or more sides of the image cube 150 and associate each side of the one or more sides with one or more directions and/or orientations (e.g., up, down, left, right, visible or not).

The image module 137 may also receive virtual or augmented image data as well. The virtual or augmented image data may comprise, for example, a virtual representation of a real-world object such as a human organ or a tool or any other object. The image module 137 may register the virtual image data to the captured image data of the image cube. For example, to output the virtual or augmented image data via the AR device 140, the image module may synchronize an orientation associated with the virtual object and an orientation associated with the image cube 150 such that a movement (e.g., a rotation, change in orientation, a change in position, a movement in real space, etc.) will be represented (e.g. output via the AR device 140) as a movement of the virtual object.

Depending on the AR application, one or more virtual objects of varying size, shape, orientation, color, and the like may be determined. For example, in a surgical application, a virtual representation of a human heart may be determined. Likewise, in an engineering application, a virtual representation of a mechanical part may be determined. In an additional exemplary application such as an AR gaming application, a virtual sword or animal may be determined. Spatial data associated with the one or more virtual objects may be determined. The spatial data associated with the one or more virtual objects may be registered to spatial data image associated with the image cube data. Registering may refer to determining the position of a given virtual object of the one or more virtual objects w/in the scene. Registering may also refer to the position of the virtual object relative any of the one or more physical objects in the augmented reality scene. The system may associate an augmented reality or virtual reality image of, for example, a body part, with the image cube. Thereby, a proceduralist may interact with the AR or VR image of the body part by manipulating the image cube.

The display 138 is an electronic device that incorporates electrically-activated components that operate to form images visible on the display. The display 138 may include backlighting (e.g. an LCD) or may be natively lit (e.g. OLED). The display 138 is shown as a single display but may actually be one or more displays. Other displays, such as augmented reality light-field displays (that project lights into three-dimensional space or appear to do so, or other types of projectors (actual and virtual) may be used.

The display 138 may be accompanied by lenses for focusing eyes upon the display 138 and may be presented as a split-screen display to the eyes of a viewer, particularly in cases in which the computing device 130 is a part of an AR device 140.

In some cases, one or more additional computing devices may be connected by the network interface 134 which may be a wired interface, such as Ethernet, universal serial bus (USB), or a wireless interface such as 802.11x, LTE, or other wireless protocol to enable the additional, computing devices to perform some or all of the operations discussed herein. For example, the CPU 131 and GPU 132 of the computing device 130 may be less powerful than that available in a connected system (e.g. a multicore process or group of multicore processors) or a group of GPUs (e.g. a single powerful GPU or a set of GPUs interconnected by SLI or CrossFire®) such that a connected computing device is better-capable of performing processor-intensive tasks. Or, a capture device (e.g. camera and associated processor and memory) in the form of a VR or AR device or simply a mobile device including a display and a camera) may be distinct from a rendering device such as a desktop computer or other computing device more-capable of performing some or all of the functions described below. In some implementations, the one or more additional computing devices may be used to perform more processor-intensive tasks, with the tasks being offloaded via the I/O interface 133 or network interface 134.

The AR device 140 may be in communication with, comprise, or otherwise be associated with the computing device 130. The AR device 140 may, itself, be a computing device, connected to a more-powerful computing device or, the AR device 140 may be a stand-alone device that performs all of the functions discussed herein, acting as a computing device 130 itself. When functioning as an augmented reality headset, the AR device 140 may incorporate an outward-facing camera that provides a real-time image of the exterior of the AR device 140 to a wearer with augmented reality objects interspersed on the display 138. Alternatively, if an AR device 140 is not present, a mobile device, or tablet, or other hand-held display and camera combination can function as a “portal” through which augmented reality or virtual reality may be seen. Although discussed generally herein in connection with an “augmented reality,” when the words “augmented reality” are used, it should be understood that this also encompasses so-called “virtual reality,” “mixed reality,” and other experiences involving the combination of any real object with a three-dimensional immersive environment or experience. As such, although AR device 140 is described as an AR headset, it is to be understood it may comprise any AR, VR, or XR technology.

The three-dimensional object 150 may comprise a physical object, placed in the world at a position or held by a user in a particular position. The three-dimensional object 150 has characteristics that are suitable for detection using computer vision techniques and, preferably, are of a type that is robust for use at different positions (e.g. close-up, arm's length, across a room), and that enable rapid detection when presented to a computing device 130 and camera 138.

The three-dimensional object 150 may be the image cube. The image may comprise one or more features that are differentiable from one another. For example, one or more shades (e.g., dark vs. light) may facilitate processing by computer vision algorithms to easily detect which side(s) are facing the camera 151. Similarly, discernible patterns may be applied to each side of the cube without having to account for more than a total of six faces. The image cube may comprise a material which has sufficient contrast so as to be imaged by a camera mounted above or adjacent to the surgical field and thereby facilitate the rendering of a virtual object. The location of the image cube in space may be determined by a variety of sensors such as computer vision sensors so as to determine an orientation of the image cube and a position in space of the image cube. For example, the c

The sides approximately correspond to up, down, left, right, forward and backward. So, when held with a face of the cube facing the user, a person's experience of the cube corresponds well, virtually and actually, with his or her experience of the real world. This makes for easier translation into an augmented reality or virtual reality environment. Three-dimensional objects of any number of sides may be used. But, cubes present unique properties that make them more-suitable to certain applications, particularly to hand-held applications. Still, when “cube” is indicated herein, any three-dimensional object of four faces or more may be substituted.

Though described as primarily passive in this application, the three-dimensional object may include its own computing device with varying levels of power, sophistication, and capabilities. In some cases, the three-dimensional object may incorporate a camera or infrared camera, lights, positional and rotational sensors, Bluetooth, RFID, WiFi and/or other systems for detecting its own position relative to an exterior room or device (e.g. the computing device 130) and for communicating that information to the computing device 130. In some cases, the three-dimensional object may take over some or all of the functions of tracking its position, rotation, and orientation relative to the computing device 130 or an environment in which it is operating (e.g. a room or external sensors, cameras, or lights).

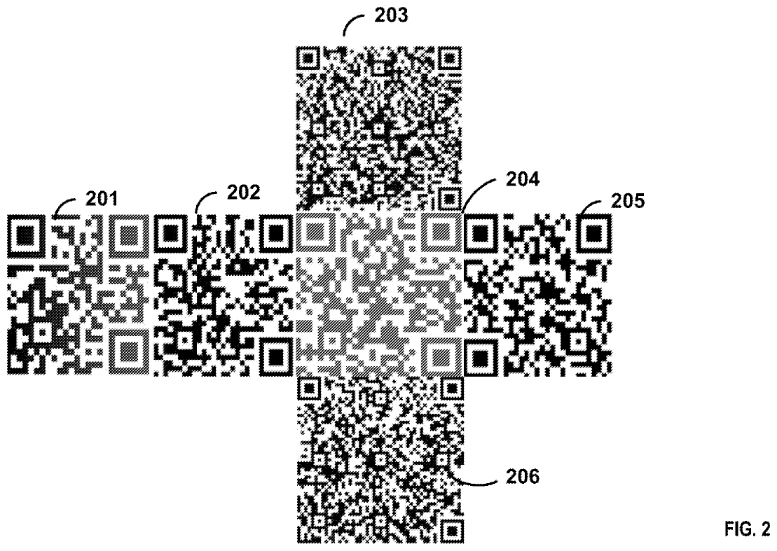

FIG. 2 is an example set of sides for a cube 200 that may be used to manipulate or interact with an augmented reality environment. FIG. 2 is merely an example of a potential cube 200. As discussed above, other shapes may be used, and virtually any type of computer-recognizable images may be used on each face. Or, as discussed above, lighting colors, depth sculpting on each face (for detection by depth-sensing systems), lighting formations (e.g. lighting in particular shapes or designs), and other detection techniques may be employed.

The cube 200 includes six faces 201, 202, 203, 204, 205 and 206. The cube is shown with its faces exploded for purposes of pointing to the characteristics of the cube 200. When formed, the cube 200 will be cubical and made from a relatively sturdy, sterilizable material. Materials may include polymers, metals, and similarly strong and resilient materials. In the cases discussed below, where electronic components are incorporated into the cube 200, it may be made of injection molded plastic, metals or other materials, so long as they are capable of wear, sterilization, and protecting those components during normal use.

Features of the cube may have relatively large-scale components that are easily distinguishable at a distance from the camera 151 (e.g. arm's length or further). While the features of cube 200 in FIG. 2 are shown as matrices, it is to be understood that any shapes or designs may be used. For instance, shapes such as circles, squares, triangles, diamonds or any other geometric shape may be used. Additionally, the features may comprise copyrightable material or any other data or information.

These features are easy for computer vision techniques to (1) detect and (2) to differentiate from one another at approximately arm's length (20-40 inches). For example, faces 201, 202, 204, and 206 include large squares as well as smaller, finer details. However, at times, a user may also move the device much closer. When held at arm's length, the intricate details of each face 201-206 may be difficult to detect. So, the large-scale images are included on each face so that computer vision techniques may use them for detection at those distances and still operate as-desired. Also, when held at close range, the details enable the computer vision to detect fine movements and to maintain stability of the image's correspondence in the virtual environment when the actual three-dimensional object is substituted in the virtual or augmented reality world for a virtual object.

However, the cube 200 may also include close-up elements for use in detection by computer vision techniques at a closer depth. When the cube 200 is held much closer to the associated, detecting camera 151, the camera 151 may not even be able to see the entirety of the large-scale images on each face and, without more, may be unable to determine which face is visible. For these cases, smaller lines and shapes are interspersed on each face of the cube 200. These may be seen in each face 201-206. And, as may be noticed, the small lines and shapes are intentionally relatively distinct from face to face. The smaller lines and shapes on each face 201-206 are presented in a variety of different rotational orientations on the respective face to facilitate quicker recognition of those lines and shapes at a variety of different viewing angles and viewing distances.

As a result, at least two detection distances are capable of detection by relatively low-resolution cameras in multiple, common lighting situations (e.g. dark, light) at virtually any angle. This technique of including at least two (or more) sizes of fiducial markers for use at different detection depths, overlaid one upon another in the same fiducial marker, is referred-to herein as a “multi-layered fiducial marker.” The use of multiple multi-layered fiducial markers makes interaction with the cube 200 (and other objects incorporating similar multi-layered fiducial markers) in augmented reality environments robust to occlusion (e.g. by a holder's hand or fingers), rapid movement, and provides strong tracking through complex interactions with the cube 200. In particular, high-quality rotational and positional tracking at multiple depths (e.g. extremely close to a viewing device and at arm's length or across a room on a table) is possible through the use of multi-layered fiducial markers.

The presence of stability from close to the camera 151 and distant from the camera 151 is unique to the use of this multi-layered fiducial marker and is noticeably different from markers employing only a single detection depth or layer. As a user moves a single-layer fiducial marker object away from the camera 151, the viewing object (e.g. camera on a computing device) has an increasingly difficult time detecting the orientation and position of the object. Or, if the object is designed for distant viewing, as it is moved closer to the camera 151, its orientation and position become increasingly difficult to track. As a result, and in either case, the object appears to move, flutter, or becomes untrackable. But, using a multi-layered fiducial marker, tracking and stability of the resulting replacement augmented reality or virtual reality object within the virtual or augmented reality world can be maintained with the object held at multiple distances from the camera 151.

In an embodiment the “light” areas of the cube 200 are raised by approximately 2-5 millimeters from the “dark” areas of each face. This may be accomplished by using injection molding wherein the raised areas, which may be dyed the lighter color or painted the lighter color or made lighter through other methods, are precisely aligned in the molding process. In this way, each of the resulting cubes 200 are identical. Alternatively or additionally, a combination or laser etching and/or powder coating may be used to create textured and/or contoured surfaces. Subsequent computer models may be based upon one of these injection-molded cubes. This is much better than the use of applied stickers, direct painting on a flat surface and other techniques because it makes the fiducial markers uniform for every cube. Thus, the computer model of each cube 200 is also uniform and image stability for the object replacing the cube within a given virtual reality or augmented reality scene is likewise uniform and without jitter present for non-injection molded three-dimensional objects.

In a typical case either a single face 201-206 is presented full-on to the camera 151 (and its associated image to a computing device for face identification) or the cube is held in such a way that multiple faces are visible to the camera 151. If the former, it is quite easy to detect which face is facing the camera because it is fully-visible to the camera 151. In the latter case, the orientation of the most front-facing face typically may be ascertained, and that information may be used in conjunction with partial views of the partially-visible sides to quickly make a very good determination which faces 201-206 are visible and their orientation.

If patterns like those of cube 200 are used, the surfaces of the cube 200 (or some surfaces—e.g. the white surfaces) may be made reflective so that they are even further contrasted with the dark portions. Or, some or all of the cube 200 may be coated in anti-reflective coating or materials so that reflections or ambient lighting does not interfere with the computer vision and detection and tracking techniques. Bright, high-contrast colors such as fluorescent colors may be used as well. Ultraviolet (for use with UV lights and UV cameras) or glow-in-the-dark paints may be used with corresponding sensors.

All of the foregoing enables finely-grained positional, orientation, and rotational tracking of the cube 200 when viewed by computer vision techniques at multiple distances from the camera 151. When held close, the object's specific position and orientation may be ascertained by computer vision techniques in many lighting situations, with various backgrounds, and through movement and rotation. When held at intermediate distances, due to the multi-level nature of the fiducial markers used, the object may still be tracked in position, orientation, through rotations and other movements. With a high level of tracking available, the cube 200 may be replaced within augmented reality scenes with other, rendered three-dimensional objects. Interactions with the cube 200 may be translated in the augmented reality environment (e.g. shown on an AR headset or mobile device) and, specifically, to the rendered object within the scene and for which the cube 200 is a real-world stand-in. Although shown as a series of high-contrast, multi-layer fiducial markers, other types of markers, such as active markers or inside-out tracking by the cube itself, or in conjunction with the computing device 130 may be used.

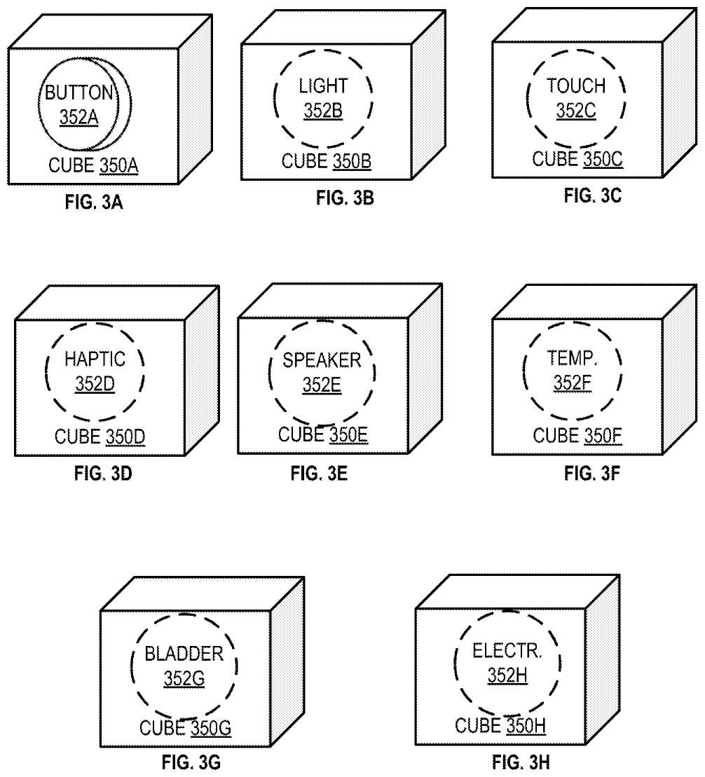

FIGS. 3A-3H, are a series of cubes, each including different elements that may be used for interactivity with an augmented reality environment. Cube 350A in FIG. 3A includes button 352A. Button 352A is shown as quite large, protruding from the exterior of cube 350A. However, button 352A may be a small button, a capacitive button, or merely an activatable switch, under the surface of the exterior of the cube 350A. Button 352A may not be a “button” at all, but instead may be a pressure detection sensor or sensors on the interior of the cube 350A that enables the cube 350A to detect when pressure of certain magnitudes is applied to the exterior of the cube 350A. The sensor(s) may be of sufficient granularity that it may detect pressure particularly on a single side of the cube 350A. As a result, interaction with the cube 350A including that pressure may be detected by (with the functionality powered by) a relatively simple processor operating within the cube 350A. That information may be transmitted from the cube 350A to an associated computing device 130 (FIG. 1).

The computing device 130 may be programmed, based upon a particular application operating, to react in a particular fashion. For example, the button 352A press or pressure sensed may operate as a “click” in a user interface. Or, the button 352A press or pressure sensed may operate as a weapon firing or object operation (e.g. door opening) within a game or other three-dimensional environment. The data may be communicated wirelessly (e.g. Bluetooth or over WiFi or RFID) between the cube 350A and an associated computing device 130 (FIG. 1).

There may be multiple buttons 352A, one or more on each face, or a series of pressure sensors accessible to the exterior of the cube 350A or within the interior of the cube 350A. Each button or pressure sensed may be associated with a particular face of the cube 350A. In this way, the interaction with a particular face through the button 352A press, or pressure sensed, may be associated with a particular interaction. Pressing on one face may enable a paintbrush tool (or a secondary interface for interacting with a tool selector), while interaction with other faces may operate to select different colors or paintbrush sizes. As discussed more fully below, translation and rotation of the cube may alternate between colors, or paintbrushes or, in other contexts, between other options within a user interface.

The button 352A may not be a button at all, but instead may be computer vision detecting the status of the face of the cube 350A. If the face is sufficiently distorted through the application of pressure, that distortion may be detected by computer vision algorithms as meeting a certain compression or distortion threshold and, as a result, a button “press” may be registered by computer vision operating on a computing device 130 (FIG. 1) without the need for any actual button within the cube 350A and, perhaps more importantly, without any electronics, battery power, or processing power incorporated into the cube 350A itself. This “button” press may operate fully on the computing device 130 while providing functionality much like that discussed above with regard to an actual, physical button or pressure sensor. Due to the details visible and not-visible on the face of the cube, computer vision techniques may even be able to localize the position of the compression on the cube face to a particular quadrant or portion of the cube. Thus, an interactive interface for each face of the cube may be created and used in the virtual or augmented reality environment without reliance upon physical buttons at all.

Cube 350B in FIG. 3B includes a light 352B and, potentially, several other lights (not labelled). The light 352B may be used for simple actions such as object tracking for computer vision applications to detect the location or orientation of the cube 350B in space in front of a camera. Then, three-dimensional virtual objects may be rendered that replace the actual, physical cube 350B in an augmented reality scene. Likewise, objects may be rendered in two dimensions on a screen. However, multiple lights, each of a different color, may be employed so as to identify particular sides or faces or edges of the associated cube 350B. As discussed above, and discussed more fully below, an easily-determinable identification of a particular face, not just the presence of an object, is useful in enabling the cube 350B to operate in conjunction with a computing device 130 (FIG. 1) to operate as a physical object that can be used to interact with a user interface presented on the display 138 of the computing device 130.

The light 352B is shown as a single light, centrally-located on a particular face. However, the light 352B may in fact be several lights, in a particular pattern around a face. Or, the light 352B may be presented to a camera in a particular form through the use of selective transparency on the face of the cube 350B or through the use of light guides. The presentation of a particular pattern, like the patterns shown in FIG. 2, may enable detection of a particular face for the cube 350B but also detection of an orientation and overall position and relative location of the cube 350B when held or placed on a table or near the computing device 130. This enables fine-grained control through translation and rotation of the cube 350B such that even small movement or rotation of the cube can be detected by computer vision techniques. Different lighting patterns or colors may be employed on each face (or both) to enable tracking and rotational detection for the interactions described herein.

The light 352B may also be dynamic such that the cube 350B incorporates a light level detector or camera to detect the light level in the room. The light 352B may react to the level of lighting in the room so that if it is very bright, the brightness of the light increases to compensate, but if the room is very dark, the brightness decreases.

Alternatively, the camera of the cube 350B or a viewing computing device 130 (FIG. 1) may detect that the background behind the cube 350B incorporates a particular color that makes it harder for the computing device to perform computer vision operations to detect the cube 350B. In response, the cube 350B may be instructed to alter the light 352B color or colors to better stand out against that background (e.g. if the background is black and white, the cube 350B may be instructed to shift to an orange and blue color palate for the lighting because orange is easier to detect against that background. If the background is detected to be very “busy”, the cube 350B may be instructed to cause the light 352B to select a uniform, simple pattern (e.g. checkers). If the background detected is very plain (e.g., one, solid color like white), the cube 350B may be instructed to present a pattern that is more complex, and that does not rely upon white at all. A multi-color LED light array may be used for this purpose and may be paired with simple processing elements within the cube 350B operating under its own instruction or instructions from an external computing device 130 (FIG. 1).

Cube 350C in FIG. 3C includes a touch interface 352C. The touch interface 352C may be a capacitive touch sensor or plate, a resistive touch sensor or plate, or some other type of touch interface. The touch interface 352C may be a single point (e.g. capable of detecting whether a touch is occurring) or may be a surface area with sufficient granularity to detect where on a surface (e.g. an entire face of the cube 350C) a touch is or touches are occurring. The touch interface 352C may be so-called” multi-touch, capable of detecting multiple simultaneous touch interactions. The touch interface 352C may be able to differentiate between a “hard” touch including more pressure than a “light” touch including less. The touch interface 352C may cover the entire surface of one or more faces of the cube 350C. The touch interface 352C is shown as only covering a portion of one face of the cube 350C, but there may be touch interfaces on each of the faces, on a subset of faces, or only on one face. The touch interface 352C may be powered by a battery and associated processor within the cube 350C.

The touch interface 352C may support interactions with faces of the cube 350C such as swipes, multi-finger swipes, mouse-like interactions, click-like interactions, or more-complex gestures along one or more surfaces of the cube 350C. For example, particular actions using the touch interface 352C may include one or more gestures performed on different faces of the cube 350C. For example, two fingers, each swiping in different directions, with each finger on a different face of the cube may instruct an associated computing device to perform one action, whereas swiping on two other faces may instruct an associated computing device to perform a different action. One set of swipes or multi-swipes or multi-clicks on two faces may switch between levels of zoom, while the same action on two different faces may select some aspect of a user interface. Actions as simple as a single touch or simultaneous touch on multiple faces may perform one action, while simultaneous touch on other faces may perform another.

For example, simultaneous touch (or simultaneous touch of sufficient detected force) on two faces opposite one another may act as a “grab” action within a three-dimensional environment to select, and “grab” onto a virtual or augmented reality object so that it may be moved or interacted with. To a user of the cube 350C, this action would “feel” a great deal like grabbing an object, for example, a catheter, surgical instrument, or anatomical structure such as tissue or bone within an augmented reality environment. During interaction with the augmented reality environment, the user may be required to maintain the opposed touches so as to maintain a “grip” on the selected or picked up object while interacting within the augmented reality environment. Holding a surgical instrument, for example, may require touches on all four faces making up one circumference of the cube (or three faces) in much the same way one might “hold” such an instrument in reality. Letting go of one or two of the four faces may cause the virtual instrument to drop from one's hand. Or, releasing one's grip to a sufficient degree—detected by the force sensors—may release an instrument, despite a “touch” being registered on all four faces or all three faces.

Cube 350D in FIG. 3D includes a haptic element 352D. The haptic element 352D may be an electric motor, including a small weight, surrounded by a coil that enables it to “vibrate” when electricity is passed through the coil so as to cause the weight within to rotate about the motor or a central axle. There are similarly linear acceleration haptic motors that intermittently charge a weight along an axis to simulate “hits” or resistance with more of a “strike” feel than a “rumble” feel. The iPhone® 6s was the first large-scale commercially-available device that incorporated a linear acceleration haptic motor in the form of its “taptic” engine. Multiple haptic elements 352D may be used for different “feels” to be emulated by the cube 350D. These are only two examples.

The haptic element 352D may operate in conjunction with an augmented reality environment generated and shown on a computing device that views the cube 350D and replaces it with some augmented reality object to better-emulate that object. For example, if a beating heart visually replaces the cube 350D on the display of a computing device viewing the cube, then the haptic element 352D may generate soft “strikes” or throbbing or vibration to emulate the associated heartbeat. The rhythm may be matched to that displayed on the display to a viewer's eyes. In such a way, the immersive experience of the associated human heart may be increased. Not only is a human heart being displayed in place of the cube 350D being held by a viewer, but the cube can be felt “beating” in that user's hand. Again, this may correspond to visual data presented on a display of the associated computing device viewing the cube 350D. Similarly, multiple virtual “objects” within the cube may be emulated through appropriate use of the haptic element 352D.

Cube 350E in FIG. 3E includes speaker 352E. The speaker 352E may be multiple speakers, one or more for each face, or may be a single speaker 352E. The speaker may be powered by battery in the cube 350E. The speaker 352E may perform actions as simple as playing music or sounds as directed by a user of an associated computing device.

However, sound may be synchronized with things taking place on the display of an associated computing device that are associated with the cube 350E. For example, if the cube 350E is replaced by an augmented reality lung, the cube may generate “exhale” or “inhale” sounds. So, as a viewer sees the augmented reality or virtual lung breathing, the sound may come from the cube itself, rather than from the mobile device, AR device or a computer speaker nearby. Virtually any type of sound created by anything that the cube is “replaced by” in the augmented realty environment may have associated sounds, noises, music, or the like. The speaker 352E on the cube 350E may make those sounds, noises or music. This, again, further increases immersion.

Cube 350F in FIG. 3F includes a temperature element 352F. The temperature element 352F may be a device that is capable of increasing or decreasing its exterior temperature, typically through the use of low electric voltage, so as to emulate the augmented reality or virtual reality object being shown on the display of an associated computing device. For example, if the cube 350F is replaced with an ice cube in the display, it would feel unusual to have that ice cube be room temperature. It would be more appropriate for that cube 350F to feel cold to the touch. The temperature element 352F may, therefore, adjust its temperature accordingly. Even if the temperature element 352F is incapable of reaching an actual freezing temperature, as an ice cube would have, even lowering the temperature appreciably would increase the immersiveness of the experience of holding a virtual reality or augmented reality ice cube. Fine grained control may or may not be possible, particularly at low voltages, but are not required to increase immersiveness.

These and many other applications of the temperature element 352F to cause the temperature of the cube 350F to better-correspond to the visual imagery being shown on the display of a viewing computing device in place of the cube 350F will cause the overall augmented reality experience of the cube 350F to be better for a user, particularly one holding the cube in their hand.

Cube 350G in FIG. 3G includes a bladder 352G. The bladder 352G may be one bladder, or multiple bladders or may not actually be a bladder, but may be a series (e.g. one for each fact or four or five for each face) of electrically-retractable and extendable elements. Similarly, one bladder or multiple bladders may be used on each face of the cube 350G. Although described as a bladder, electromagnetic actuators, levers, electronic pistons, and other, similar, systems may also be used.

The bladder 352G may be controlled by electronics on the cube 350G in concert with instructions from the computing device to either fill or empty the bladders (or to extend or contract the electronic elements) to cause the cube 350G to deform. This deformation may be controlled by the computing device to better-correspond to the shape of the object being displayed on the computing device.

For example, if a virtual or augmented reality heart is displayed on the computing device display, a series of 6 bladders 352G, one for each face, may all be inflated to cause the cube to be more round. As a result, the cube 350G feels more like a heart and less like the cube. As discussed above, haptic element 352D may simultaneously generate heart “beats” that are felt in the more round cube 350G to increase the overall similarities of the virtual and actual experience.

Cube 350H in FIG. 3H includes an electrode 352H. This electrode 352H is labeled as though it is a single electrode, but it may in fact, be a series or a multiplicity of electrodes or similar electric elements on each face of the cube 350H or with multiple electrodes on each face of the cube 350H. Research into particular voltages applied to electrodes particularly to small electrodes, has indicated that at certain voltages, applied directly to the skin, the nerve endings associated with touch, pressure, heat, or pain can be stimulated in such a way to emulate very similar experiences by causing the desired nerves to react, without actually causing the desired feeling (e.g. touch, pressure, heat, pain, etc.).

So, small electrical currents may be passed through a user's hand or to the skin of a user's hand, while holding the cube 352H to simulate a particular “feel” of the cube 350H through only the use of a small current. This current may simulate texture (e.g. fur, spikes, cold stone or metal, and the like) through the application of an appropriate voltage. Thus, the electrode 352H (or multiple electrodes) may be used to emulate a broad array of experiences for a holder of the cube 350H.

Though each of the cubes 350A-350H are discussed in turn, any of the various elements discussed may be combined with one another in a single cube 350. So, haptic elements 352D may be combined with touch interface 352C and/or may be combined with electrode 352H and so on. Each of the elements were discussed individually so as to inform as to their intended uses, but combination uses may also be made. Likewise, each of the elements can be provided on one or up to all six faces of the cube, or in a combination such as touch interface 352C and light 352B on each face, or any other permutation. Each of these options available for application by the cube to interact with a holder of the cube, may be described as “dynamics.” Dynamics, as used herein, is similar to haptics, but is intentionally a broader term incorporating the use of one or more of the elements 352A-352H discussed above to create an overall dynamic experience to a holder of the cube. As such, the various elements 352A-352H may be termed “dynamic elements.”

For example, while gripping the cube as detected by the touch interface 352C and using an augmented reality sword to strike virtual enemies, the haptic element 352D may react with an appropriate “thud” or “impact” feeling in response to each strike. This may further engage immersion of one wielding the “virtual” weapon. Similarly, audio feedback associated with a gun firing may be generated by speaker 350 every time button 352A is pressed (or pressure is sensed) to better-emulate a gun firing. The temperature element 352F may heat up as a gun is rapidly fired for a time to feel more like a real gun heating up in response to rapid firing. Likewise, bladder 352G may alter the shape of the cube 350 to better-feel like the handle of a pistol. Though these examples are made with reference to a weapon-based game, virtually any other options are available, so long as the associated elements are capable of emulating or somewhat emulating a particular augmented reality object through clever utilization of one or more elements.

Communication between a computing device and the cube 350 may take place using Bluetooth®, WiFi, near field, RFID, infrared or any other communication protocol that is appropriate given the bandwidth and power-consumption requirements.

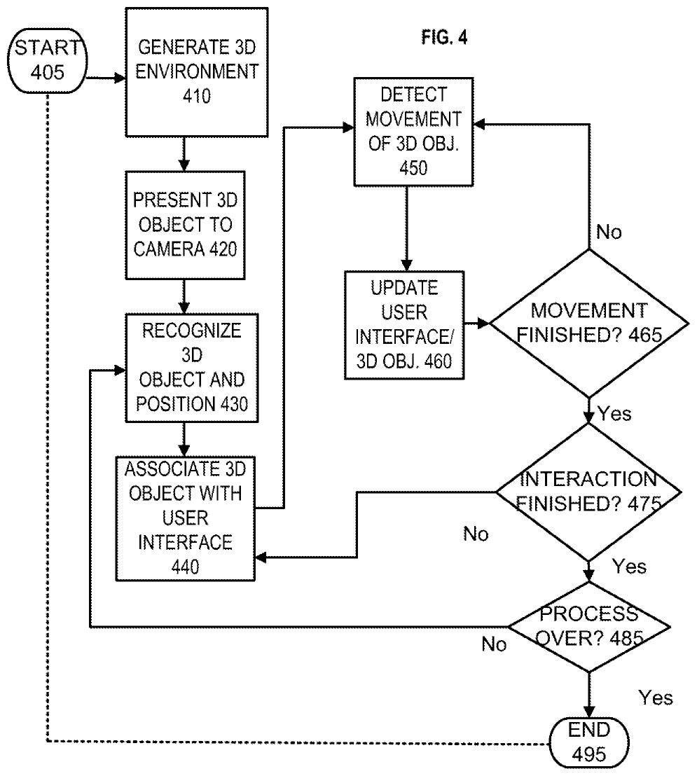

Referring now to FIG. 4, a flowchart for a method for interacting with an augmented reality environment is shown. The flow chart has both a start 405 and an end 495, but the process is cyclical in nature, as indicated by the dashed return arrow. The process may take place many times while a computing device is viewing and tracking the cube or other three-dimensional object.

Following the start 405, the process begins with the generation of a three-dimensional environment at 410. This environment is generated on the display of a computing device. The three-dimensional environment may entirely replace reality (e.g. a virtual reality environment) or may supplement reality with “augmentations” (e.g. augmented reality) or may only incorporate one or more particular elements (including simple rendering on a 2-dimensional screen). This replacement and/or supplementation takes the form of a three-dimensionally-rendered environment or objects within the environment. So, for example, a user in virtual reality may suddenly appear, visually, to be present on the Temple Mount in Jerusalem or along the shore of Lake Como in Italy or in a completely fictional location within an immersive game, a story-based environment, or other location.

A user in augmented reality typically remains present in their current location with a camera built into an augmented reality headset or device (e.g. a mobile phone, screen mounted in the operating room) acting as a “window” into the augmented reality world. Within the augmented reality word, the user may see primarily his or her current location, but additional objects, persons, or other elements may be added. So, one may be sitting in his or her office, but when looking through the augmented reality computing device, a fairy may be floating near a wall within the office or a narrator may be standing in a nearby hallway narrating to the user of the augmented reality device. Augmented reality typically tries to merge the real and un-real to appear as normal as possible, but more cartoon-like or game-like experiences are also possible. To this end, more-advanced augmented and virtual reality systems rely upon lidar, infrared cameras and scanners, and other, similar technology, to physically map the three-dimensional characteristics of the present environment. In this way, the precise size and shape of a room may be ascertained and any augmented reality objects, people, or other elements may be integrated more accurately. For example, images may replace actual walls without “turning corners” or appearing to hang in mid-air. People can be properly presented when behind furniture so that perspective does not appear to have been violated. These and other capabilities are possible, depending on the robustness of the associated computing device that is rendering the three-dimensional environment.

In this context, most augmented reality or virtual reality environments in the present state of the art have relied primarily, if not exclusively, upon visuals. Some more sophisticated systems also incorporate controllers that are capable of being tracked, either by the headset itself or by external trackers. In this way, systems like the PSVR®, for example, can track controllers held in the hands of users. Those controllers have buttons on them that enable some basic interactivity. However, the tracking for PSVR®, systems, for example, follows light emitted by a single spherical ball of a unique color (so multiple balls may be tracked simultaneously). Each “ball” does not have a side, and up or a down, precisely because they are round. Their location, but not orientation, may be tracked.

Similarly, the Oculus® Touch® controllers incorporate buttons and an exterior, circular loop surrounding the hands of a holder that emits infrared light that may be tracked. In this way, a holder's hand positions, and orientations may be tracked. However, the functionality for that tracking requires an external (or several) cameras to track the motion of those hand-held controllers.

In contrast, the next step of using the cube described herein is to present the cube (or other three-dimensional object) to the camera 420 of the computing device. In the most common case, this camera will be the camera on a mobile device (e.g. an iPhone®) that is being used as a “portal” through which to experience the augmented reality environment. The camera has none of the accoutrements of complex systems like the Oculus® Touch®. Instead, it is merely a device that most individuals already have in their possession and that includes no specialized hardware for detection of particular infrared markers or other specialized elements. Another common use will be to have a camera mounted above or adjacent to the surgical operating field, which registers and tracks the movements of the cube and then passes this information through software that renders images on a two-dimensional screen visible to the proceduralist who is manipulating the cube.

Likewise, though the three-dimensional object is described above as capable of incorporating a multiplicity of elements that may augment an immersive experience, it may, instead, be as simple as the cube with six unique fiducial markers. Objects with as few as two or three unique fiducial markers may suffice. As used herein, the phrase “unique fiducial marker” expressly does not include multiple single lights, infrared or otherwise, used as a set as a fiducial marker. In the understanding of this description, an entire controller, such as the Oculus® Touch® that utilizes a series of lights is, effectively, one fiducial marker. Without several lights in known positions (and typically many more) computer vision techniques could not know position, orientation, or relative location of the Oculus® Touch® controller. Thus, a single light on the Oculus® Touch® is not a fiducial marker at all—it is a light. Multiple lights, together, make up a single unique fiducial marker as that phrase is used in this disclosure.

Discussed another way, the phrase “unique fiducial marker” means an individual marker, complete in itself, that can be used to distinguish one face or one entire edge (not a single point) of a controller or three-dimensional object from another face or edge. In addition, a unique fiducial marker may be used, in itself, to determine the position of the object bearing the fiducial marker. As seen in this application, one way of doing that is to create a six-sided cube with each side bearing a unique fiducial marker. The Oculus® Touch® and other, similar, AR and VR controllers rely upon a known configuration for infrared lights on the controller. While accurate, each of these lights alone is not “complete in itself” in that a single light is insufficient to distinguish one face or one edge of an Oculus® Touch® controller from another. In a group, collectively, they may be used to derive orientation and position information, but even only two of the lights, alone, do not define any face or edge.

The use of unique faces, each including a unique fiducial marker, is uniquely important because it lowers the overall investment necessary to experience immersive virtual or augmented reality incorporating a “controller” and enables additional functions not available without the expense of more-complex VR and AR devices or systems and controllers.

Though discussed herein as a multi-layered, unique fiducial marker that is in the form of a black and white, high-contrast image on the face of the three-dimensional object; in some cases, other computer detection techniques may be used for some aspects of the positional, rotational, and orientation tracking of the three-dimensional object. For example, unique fiducial markers may be edge or corner detection techniques such as each edge or corner of a three-dimensional object bearing a unique color or colors. A combination of a specific set of unique colors, one on each corner, may be used to determine a specific face associated with those edges, and to determine the orientation (e.g. the orange corner is bottom right of the cube, and the purple corner is top left, therefore the cube is in this orientation and at this distance based upon the sizes of the corner colors detected).

Likewise, the colors or markers may be passive or active, including paint, reflective materials and the like or reliant upon lights or interior lights that escape from the surface of the three-dimensional object only in certain orientations and/or patterns and/or colors. For example, the unique, multi-layered fiducial markers may be only white and black, but the white may be generated by lights passing through the exterior of the three-dimensional object. Alternatively or in addition, the lights may be color coded such that each face is a unique colored light, but the pattern may be the same on each face or corner. Alternatively, the pattern may be different on each face or corner, but the colors may be the same.

Similarly, other techniques may be used, at least in part, for detection of the position, orientation, and rotation of the three-dimensional object. Those include outside in tracking for the three-dimensional object (e.g. the object includes cameras or marker detectors for tracking its own position and associated communication capabilities with external devices), light-based detection, the use of multiple, exterior cameras to detect more than one or a few sides simultaneously. Motion and rotational and gravitational sensors may be included in the three-dimensional object itself to track or to enhance tracking of the three-dimensional object.

Next, the three-dimensional object is recognized by the camera of the computing device at 430 while the position, orientation, and motion begin being tracked. At this stage, not only is the three-dimensional object recognized as something to be tracked, but the particular side, face, or fiducial marker (and its orientation, up or down or left or right) is recognized by the computing device. The orientation is important because the associated software also knows, if a user rotates this object in one direction, which face will be in the process of being presented to the camera of the computing device next and can cause the associated virtual or augmented reality rendered object to react accordingly. At 430, the tracking, position, orientation and motion (including rotation) begin being tracked by the software in conjunction with the camera. As discussed above, the camera may be used to perform this tracking, but the object may self-track and report its position, orientation, and motion to an associated computing device. Or, alternatively, the object and computing device may both perform some or all of the processes involved in tracking.

Now, the three-dimensional object (e.g. cube) may be associated with some aspect of the user interface of the augmented realty or virtual reality environment being shown on the display. This association may be as simple as “you” (the user of the computing device) are the three-dimensional object within a virtual or augmented reality environment being shown on the computing device. Or, the three-dimensional object may be a stand-in for a surgical instrument or other type of object. Or, the three-dimensional object may be associated with a particular menu, operation, volume change setting, the user's “view” or perspective of the augmented reality environment, a page of a virtual or augmented reality book, and other similar aspects of a virtual or augmented reality environment or object.

That association may take place automatically. For example, a proceduralist may load a particular surgical scenario, application, or experience. Upon load, the surgical scenario, application, or experience may begin using the camera of the computing device. The surgical scenario, application, or experience may be expecting to see the cube or other three-dimensional object. So, it may continually scan for objects within the frame of the camera that could be the expected three-dimensional object. Once found, the software may automatically associate the three-dimensional object with a particular aspect of the user interface.

For example, the object may become a heart, floating in space, and movement of that object may cause the heart to move in a similar fashion, mirroring the actions of the user on the object. Rolling the object forward may cause the heart to turn to expose the ventral side or to become transparent, revealing internal structures. Rolling the object backward may cause the heart to expose its dorsal side or to become opaque, no longer showing internal structures.

In other cases, the association may be manually-selected (e.g. through interaction with a menu on the display of the computing device) or may be enabled through interaction with the three-dimensional object itself. For example, clicking, squeezing, or moving the object in a particular fashion (e.g. to spell a “Z” in the air) may cause the object to take control over a “zoom” function within the interface or to take control over the audio volume of the associated application, or to select a paintbrush within an application. The actions and or movement may be previously-determined by the application itself or may be user-programmable. In this way, the object may act as a “mouse” or as some other interactive element for any number of applications. For example, a click, and a twist (rotation around a Y axis) may cause the object to act (and to visually appear in the display of the associated application) as a volume knob. As it is turned to the right, audio volume may increase. As it is turned to the left, volume may decrease, in much the same fashion as a typical volume knob, all the while the user is actually merely holding the cube with six-faces including different fiducial markers.

Once the three-dimensional object is associated with a particular user interface element at 440, movement of the object may be detected at 450. This movement may be in essentially any form. For example, translational movement may be “away from” a user (or the display or camera) or toward the user, in a rotation about an axis, in a rotation about multiple axes, to either side or up or down. The movement may be quick or may be slow (and that may be detected and may matter, depending on the function or augmented reality object associated with the three-dimensional object).

The movement may also be kinetic, such as when the object is thrown up in the air, between users, or at a target. Due to the capability of simple computer vision techniques to track the three-dimensional object at multiple depts. (e.g. the multi-layer fiducial markers), the object may be reliably tracked at distances close to a user before being thrown, and further from a user, after being thrown. Multiple three-dimensional objects may be used in some cases as part of games where throwing or passing objects is done.

Since generalized object tracking has existed for some time, the most relevant movement for purposes of this application are those that involve tracking of a particular face or faces of the three-dimensional object. Most commonly, that will be rotation about one or more axes. However, it may also be tracking which “face” is currently being compressed, clicked, or which face is being held in a particular user's hand (or where). For example, detecting that face x is visible, and assuming that the three-dimensional object is being held in a right hand, the face y may be most-likely to be held closest to the skin of a user's hand. That information may be used to provide dynamics to that face or closest to that face (e.g. heat, or a strike, or the like) when interactions with the object take place in the virtual or augmented reality environment.

The detected movement may be used to update the user interface and/or the three-dimensional object itself 460. In particular, the associated three-dimensional object with user interface 440 step may be used as a preliminary step to identify the aspect of the user interface, automatically or as a selective action, which will be the subject of the update of the user interface and/or three-dimensional object at 460. So, for example, a volume interaction may be selected at 440, in which case motion detected at 450 may be use to update the volume. Or, if a color selector is associated at 440 with the three-dimensional object, then rotation of the three-dimensional object detected at 450 may result in a color change (e.g. for a paint brush being used by a user and/or represented by the three-dimensional object within the augmented realty or virtual reality environment) for the paint being used. If the three-dimensional object is associated with an anatomic organ or surgical tool or other relevant object in a virtual reality or augmented reality environment at 440, then the detected movement at 450, for example rotation forward, may cause that augmented reality or virtual reality object to rotate or become transparent or to become larger or smaller or perform other actions.

At decision step 465 a determination whether the particular movement is finished is made by the associated computing device tracking the movement of the three-dimensional physical object. This may be through a deselection or completed selection by the three-dimensional object through an action (e.g. click, or swipe or similar action) or may be through a timeout (e.g. 4 seconds elapse without change, then a particular action or user interface element is selected). If the particular movement is not finished (“no” at 465), then the process continues to detect the movement to 450.

If the particular movement is finished (“yes” at 465″), then the process continues to determine if the overall interaction is finished at decision step 475. Here, the application, training or rehearsal environment, operative theatre environment or other virtual or augmented reality environment operating as software on the computing device may check whether the overall process is complete. This may be simple, e.g. the encounter is over or the user is no longer navigating through the object or the like. However, it may also be complex, such as a user has de-selected the paint brush tool within the paint-like application, but has not yet exited the application. If this is the case (“no” at 475), then the computing device may associate the three-dimensional object with some other aspect of the user interface at 440 and the process may begin again. For example, the user has de-selected the paintbrush, but has now selected the paint sprayer tool. The overall process is not complete, but the particular interaction being tracked initially has ended.

If the interaction has ended (“yes” at 475), then the computing device may determine whether the overall process is over at decision step 485. At this step, the software may simply be closed, or the mobile device or other computing device be put away. If so (“yes” at 485), then the process is complete at end point 495. If not, (“no” at 485), then the three-dimensional object may have been lost through being obscured to the camera, may have moved out of the field of view or may otherwise have been made unavailable. The process may continue with recognition of the object and its position at 430 and the process may continue from there.

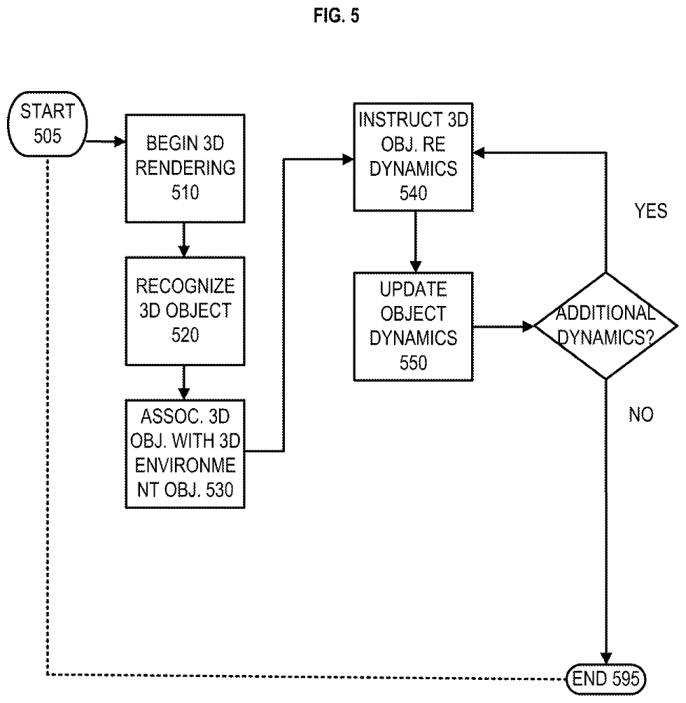

FIG. 5 is a flowchart for a process of updating dynamics of a three-dimensional object in response to changes in an augmented reality environment. The flow chart has both a start 505 and an end 595, but again the process is cyclical in nature as indicated. The process may take place many times while a computing device is viewing and tracking the cube or other three-dimensional object.

The process begins with rendering a three-dimensional environment (or object) such as a virtual reality or augmented reality environment at step 510. This is discussed above. The rendering device may be a computing device such as an AR headset, a mobile device, a tablet, a mounted screen in the operating room or the like.

At step 520, the computing device may be presented with a three-dimensional object and may recognize it as such. As discussed above, the object may include one or more fiducial markers, lighting, or other aspects that enable it to be recognized. For the purposes of engendering dynamics to a three-dimensional object, that object need not necessarily have multiple fiducial markers, but it may.

The three-dimensional object may then be associated with a three-dimensional environmental object at step 530. So, within the virtual or augmented reality, the object may be associated, automatically, or through user action/selection, with an object. At this point, the actual, real three-dimensional object, being viewed on the display of the computing device, may be substituted on that display for an augmented reality or virtual reality object (e.g. a heart, a surgical instrument, a personal avatar, etc.) In an augmented reality environment, the rest of the reality would continue to be displayed normally, but the object (e.g. heart) would appear to be being held in the user's hand as opposed to the cube or other three-dimensional object.

The computing device may be in communication (e.g. via Bluetooth® or otherwise) with the three-dimensional object which incorporates one or more of the elements discussed with reference to FIG. 3, above, that are capable of generating dynamics. At 540, the augmented reality heart may begin “beating” on the display of the computing device. Simultaneously, the haptic element 352D may be instructed by the computing device to begin “beating” or operating so as to emulate beating of the heart that matches the rhythm of that being displayed on the display. Still further, the temperature element 352F may be instructed to raise the temperature of the three-dimensional object slightly to better-emulate a human heart. Finally, the bladder 352G may be instructed to inflate all bladders to feel more “round” so as to feel more like a human heart when held in the user's hand.

At 550, the dynamics of the three-dimensional object are updated as instructed at 540. As discussed above, virtually any combination of dynamics may be employed together to generate different sensations or feelings for a user, particularly a user holding the three-dimensional object.

If any additional dynamics are desired (“yes” at decision step 555) (e.g. the heart ceases beating in a dramatic fashion to demonstrate a cardiac arrest), then the instructions may be received from software operating on the computing device at 540 and the object dynamics may be updated again at 550.

If no further dynamics are to be updated (“no” at 555), then the process may end at 595 until the next iteration of object dynamics is desired.

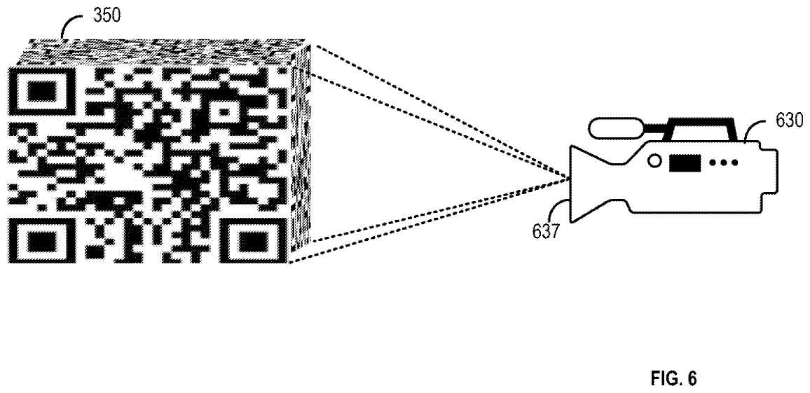

FIG. 6 is an example of a computing device 630 engaged in computer vision detection and tracking of a three-dimensional object 650. The computing device 630 is shown as the back of a mobile device or the front face of an augmented reality or virtual reality headset. The computing device 630 includes a camera 637 that is capturing images in front of the computing device 630. One of those objects in front of the computing device 630 is the three-dimensional object 650. The three-dimensional object may be a six-sided cube including unique fiducial markers on each face so that its orientation, in addition to position, may be tracked by the camera 637.

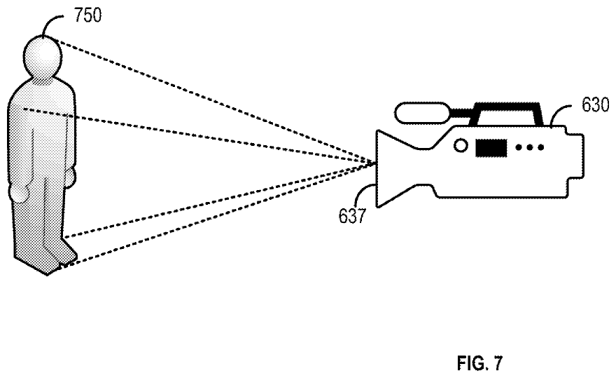

FIG. 7 is an example of a computing device 730 substituting a detected three-dimensional object 650 (FIG. 6) in an augmented reality environment for a rendered three-dimensional object 750, such as a person. FIG. 7 is identical to FIG. 6 and the description of the associated elements will not be repeated here, except to point out that the computing device 730 is replacing the three-dimensional object 650 of FIG. 6 in a rendered environment with the rendered three-dimensional object 750. The rendered three-dimensional object 750 may be rendered in exactly the same position and orientation as the three-dimensional object 650. And, as discussed below, the rendered three-dimensional object 750 may move in the same way as the three-dimensional object 650 is moved.

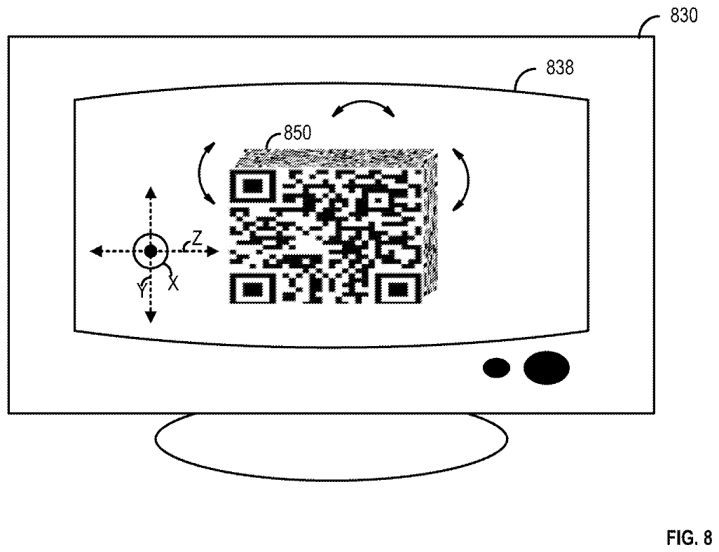

FIG. 8 is an on-screen display 838 of a computing device 830 showing a three-dimensional physical object 850 capable of rotation about three axes. The three-dimensional physical object 850, detected by camera 737, may appear on the display 838. Because the object 850 has unique fiducial markers on each face, its orientation may be detected and multiple sides are typically seen at once. Rotation and orientation may be tracked using only an image camera 737 (e.g. RGB, black and white, or ultraviolet).

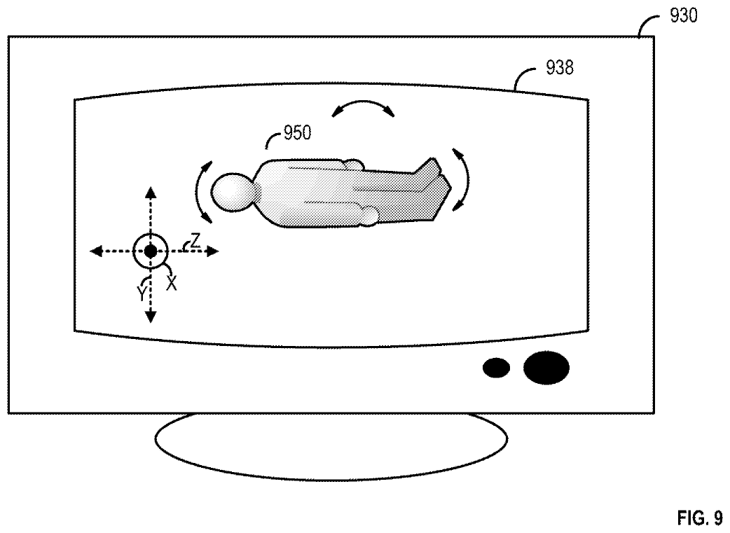

FIG. 9 is an on-screen display 938 of a computing device 930 showing a substitution of a rendered three-dimensional object 950 in place of a physical three-dimensional object 850. Here, the rendered three-dimensional object 950 on the display 938 replaces the actual three-dimensional object 850 being captured by the camera 737. The display 938 may present reality or a virtual environment in which the rendered three-dimensional object 950 is placed. And, the rotation may be tracked, along with the other functions described as taking place herein.

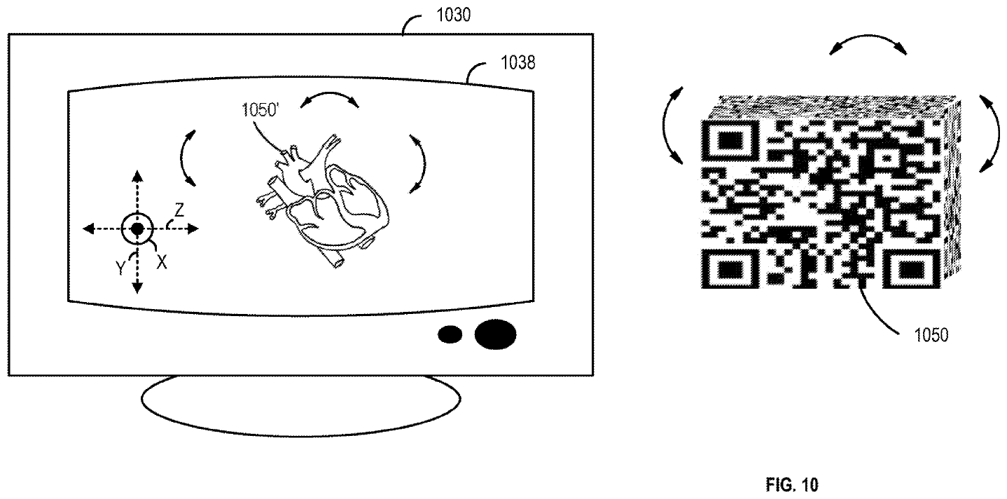

FIG. 10 is an example of a rendered object 1050′ substituting for a three-dimensional physical object 1050 in an augmented reality display 1038, the three-dimensional physical object 1050 incorporating dynamics associated with the rendered object 1050′.

As discussed above, the dynamics may be any number of things or a group of things generated by the various elements 352A-352H (of FIG. 3). The dynamics of the heart shown as the rendered three-dimensional object 1050′ may include the heartbeat, the heat, the roundedness of the cube based upon shape forming bladders. As a result, the real world three-dimensional physical object 1050 may “feel” in a manner similar to the rendered three-dimensional object's 1050′ appearance on the display 1038. The dynamics may be updated to correspond to the object or to provide feedback for other interactions with the environment shown on the display 1038.

Throughout this description, the embodiments and examples shown should be considered as exemplars, rather than limitations on the apparatus and procedures disclosed or claimed. Although many of the examples presented herein involve specific combinations of method acts or system elements, it should be understood that those acts and those elements may be combined in other ways to accomplish the same objectives. With regard to flowcharts, additional and fewer steps may be taken, and the steps as shown may be combined or further refined to achieve the methods described herein. Acts, elements and features discussed only in connection with one embodiment are not intended to be excluded from a similar role in other embodiments.

As used herein, “plurality” means two or more. As used herein, a “set” of items may include one or more of such items. As used herein, whether in the written description or the claims, the terms “comprising”, “including”, “carrying”, “having”, “containing”, “involving”, and the like are to be understood to be open-ended, i.e., to mean including but not limited to. Only the transitional phrases “consisting of” and “consisting essentially of”, respectively, are closed or semi-closed transitional phrases with respect to claims. Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. As used herein, “and/or” means that the listed items are alternatives, but the alternatives also include any combination of the listed items.

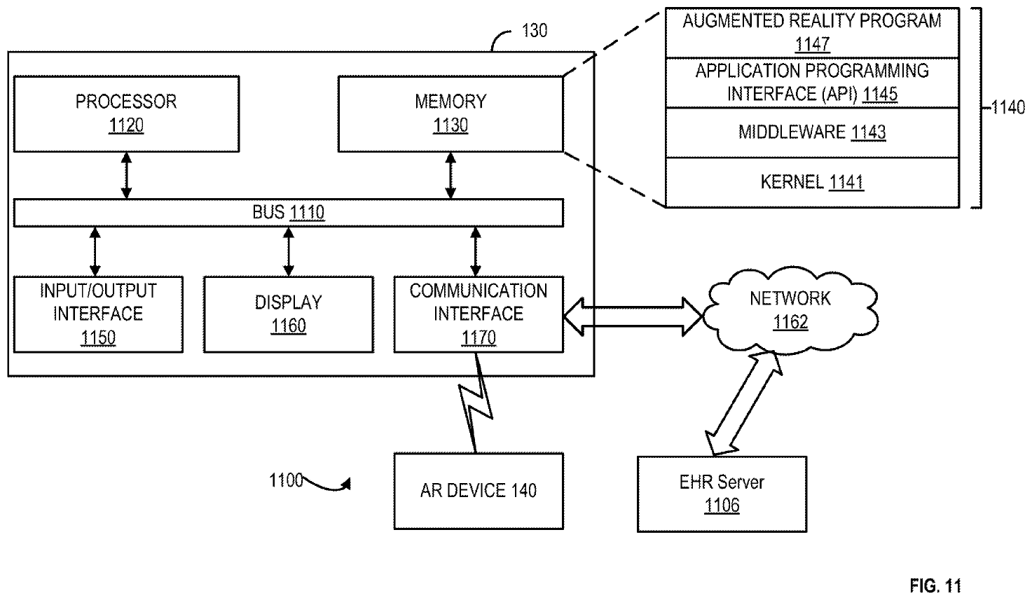

FIG. 11 illustrates a network environment including the computing device 130 configured for augmented reality applications according to various embodiments. Referring to FIG. 11 the computing device 130 in the network environment 1100 is disclosed according to various exemplary embodiments. The AR device 140 may include a bus 1110, a processor 1120, a memory 1130, an input/output interface 1150, a display 1160, and a communication interface 1170. In a certain exemplary embodiment, the computing device 130 may omit at least one of the aforementioned constitutional elements or may additionally include other constitutional elements. The computing device 130 may be, for example, a tablet computer, a laptop, a desktop computer, a smartwatch, and the like.

The bus 1110 may include a circuit for connecting the aforementioned constitutional elements to each other and for delivering communication (e.g., a control message and/or data) between the aforementioned constitutional elements.

The processor 1120 may include one or more of a Central Processing Unit (CPU), an Application Processor (AP), and a Communication Processor (CP). The processor 1120 may control, for example, at least one of the other constitutional elements of the AR device 140 and may execute an arithmetic operation or data processing for communication. The processing (or controlling) operation of the processor 1120 according to various embodiments is described in detail with reference to the following drawings.

The memory 1130 may include a volatile and/or non-volatile memory. The memory 1130 may store, for example, a command or data related to at least one different constitutional element of the computing device 130. According to various exemplary embodiments, the memory 1130 may store a software and/or a program 1140. The program 1140 may include, for example, a kernel 1141, a middleware 1143, an Application Programming Interface (API) 1145, and/or an augmented reality program 1147, or the like.

At least one part of the kernel 1141, middleware 1143, or API 1145 may be referred to as an Operating System (OS). The memory 1130 may include a computer-readable recording medium having a program recorded thereon to perform the method according to various embodiment by the processor 1120.

The kernel 1141 may control or manage, for example, system resources (e.g., the bus 1110, the processor 1120, the memory 1130, etc.) used to execute an operation or function implemented in other programs (e.g., the middleware 1143, the API 1145, or the application program 1147). Further, the kernel 1141 may provide an interface capable of controlling or managing the system resources by accessing individual constitutional elements of the computing device 130 in the middleware 1143, the API 1145, or the augmented reality program 1147.

The middleware 1143 may perform, for example, a mediation role so that the API 1145 or the augmented reality program 1147 can communicate with the kernel 1141 to exchange data.

Further, the middleware 1143 may handle one or more task requests received from the augmented reality program 1147 according to a priority. For example, the middleware 1143 may assign a priority of using the system resources (e.g., the bus 1110, the processor 1120, or the memory 1130) of the computing device 130 to the augmented reality program 1147. For instance, the middleware 1143 may process the one or more task requests according to the priority assigned to the augmented reality program 1147, and thus may perform scheduling or load balancing on the one or more task requests.

The API 1145 may include at least one interface or function (e.g., instruction), for example, for file control, window control, video processing, or character control, as an interface capable of controlling a function provided by the augmented reality program 1147 in the kernel 1141 or the middleware 1143.

For example, the input/output interface 1150 may play a role of an interface for delivering an instruction or data input from the user 500 or a different external device(s) to the different constitutional elements of the computing device 130. Further, the input/output interface 1150 may output an instruction or data received from the different constitutional element(s) of the AR device 140 to the different external device.

The display 1160 may include various types of displays, for example, a Liquid Crystal Display (LCD) display, a Light Emitting Diode (LED) display, an Organic Light-Emitting Diode (OLED) display, a MicroElectroMechanical Systems (MEMS) display, or an electronic paper display. The display 1160 may display, for example, a variety of contents (e.g., text, image, video, icon, symbol, etc.) to the user 500. The display 1160 may include a touch screen. For example, the display 1160 may receive a touch, gesture, proximity, or hovering input by using a stylus pen or a part of a user's body.

The communication interface 1170 may establish, for example, communication between the AR device 140 and an external device (e.g., the mobile device 201, a microphone/headset 1102, or the EHR server 1106) through wireless communication or wired communication. For example, the communication interface 1170 may communicate with the EHR server 1106 by being connected to a network 1162. For example, as a cellular communication protocol, the wireless communication may use at least one of Long-Term Evolution (LTE), LTE Advance (LTE-A), Code Division Multiple Access (CDMA), Wideband CDMA (WCDMA), Universal Mobile Telecommunications System (UMTS), Wireless Broadband (WiBro), Global System for Mobile Communications (GSM), and the like. Further, the wireless communication may include, for example, a near-distance communication. The near-distance communication may include, for example, at least one of Wireless Fidelity (WiFi), Bluetooth, Near Field Communication (NFC), Global Navigation Satellite System (GNSS), and the like. According to a usage region or a bandwidth or the like, the GNSS may include, for example, at least one of Global Positioning System (GPS), Global Navigation Satellite System (Glonass), Beidou Navigation Satellite System (hereinafter, “Beidou”), Galileo, the European global satellite-based navigation system, and the like. Hereinafter, the “GPS” and the “GNSS” may be used interchangeably in the present document. The wired communication may include, for example, at least one of Universal Serial Bus (USB), High Definition Multimedia Interface (HDMI), Recommended Standard-232 (RS-232), power-line communication, Plain Old Telephone Service (POTS), and the like. The network 1162 may include, for example, at least one of a telecommunications network, a computer network (e.g., LAN or WAN), the internet, and/or a telephone network.

According to one exemplary embodiment, the EHR server 1106 may include a group of one or more servers. The EHR server 1106 may be configured to generate, store, maintain, and/or update various data including electronic health records, image data, location data, spatial data, geographic position data, and the like and combinations thereof. The EHR server 1106 may electronic health records associated with a patient.

In an embodiment, the AR device 140 may be configured to execute the augmented reality program 1147. Using the various sensors and modules, the AR device 140 may a virtual representation of a physical object. In an embodiment, the AR device 140 may use a combination of sensors to detect objects within a field of view. In an embodiment, the AR device 140 may use a combination of sensors to image data or orientation information about the AR device 140, the one or more physical objects or the virtual object. In an embodiment, the AR device 140 may detect the presence of the one or more physical objects within the field of view 600, as well as the position of the one or more physical objects and the various distances between the one or more physical objects and the AR device 140.

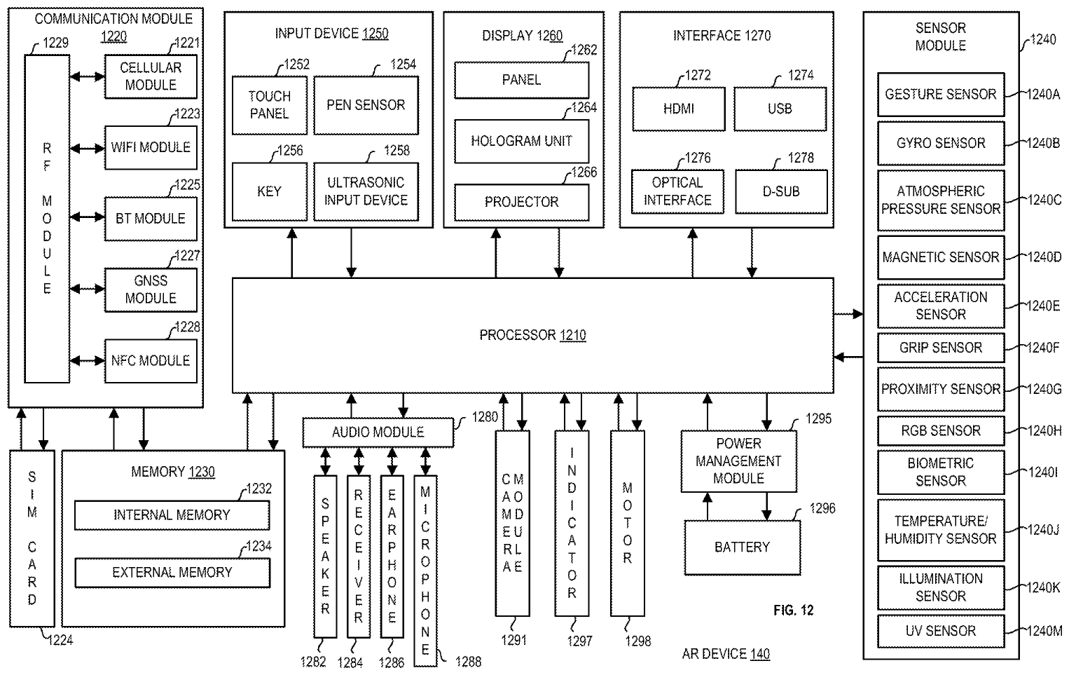

FIG. 12 is a block diagram of an AR device 140 according to various exemplary embodiments. The AR device 140 may include one or more processors (e.g., Application Processors (APs)) 1210, a communication module 1220, a subscriber identity module 1224, a memory 1230, a sensor module 1240, an input unit 1250, a display 1260, an interface 1270, an audio module 1280, a camera module 1291, a power management module 1295, a battery 1296, an indicator 1297, and a motor 1298. Camera module 1291 may comprise an aperture configured for a change in focus.

The processor 1210 may control a plurality of hardware or software constitutional elements connected to the processor 1210 by driving, for example, an operating system or an application program, and may process a variety of data including multimedia data and may perform an arithmetic operation (for example, distance calculations). For instance, the processor 1210 may be configured to generate a virtual object, and place the virtual object within an augmented reality scene. The processor 1210 may be implemented, for example, with a System on Chip (SoC). According to one exemplary embodiment, the processor 1210 may further include a Graphic Processing Unit (GPU) and/or an Image Signal Processor (ISP). The processor 1210 may include at least one part (e.g., a cellular module 1221) of the aforementioned constitutional elements of FIG. 12. The processor 1210 may process an instruction or data, for example the augmented reality program 1147, which may be received from at least one of different constitutional elements (e.g., a non-volatile memory), by loading it to a volatile memory and may store a variety of data in the non-volatile memory. The processor may receive inputs such as sensor readings and execute the augmented reality program 1147 accordingly by, for example, adjusting the position of the virtual object within the augmented reality scene.

The communication module 1220 may include, for example, the cellular module 1221, a Wi-Fi module 1223, a BlueTooth (BT) module 1225, a GNSS module 1227 (e.g., a GPS module, a Glonass module, a Beidou module, or a Galileo module), a Near Field Communication (NFC) module 1228, and a Radio Frequency (RF) module 1229. The communication module may receive data from the camera and/or the EHR server 1106. The communication module may transmit data to the display and/or the EHR server 1106. In an exemplary configuration, the AR device 140 may transmit data determined by the sensor module 1240 to the display and/or the EHR server 1106. For example, the AR device 140 may transmit, to the display, via the BT module 1225, data gathered by the sensor module 1040.

The cellular module 1221 may provide a voice call, a video call, a text service, an internet service, or the like, for example, through a communication network. According to one exemplary embodiment, the cellular module 1221 may identify and authenticate the AR device 140 in the network 762 by using the subscriber identity module (e.g., a Subscriber Identity Module (SIM) card) 1224. According to one exemplary embodiment, the cellular module 1221 may perform at least some functions that can be provided by the processor 1210. According to one exemplary embodiment, the cellular module 1221 may include a Communication Processor (CP).

Each of the WiFi module 1223, the BT module 1225, the GNSS module 1227, or the NFC module 1228 may include, for example, a processor for processing data transmitted/received via a corresponding module. According to a certain exemplary embodiment, at least some (e.g., two or more) of the cellular module 1221, the WiFi module 1223, the BT module 1225, the GPS module 1227, and the NFC module 1228 may be included in one Integrated Chip (IC) or IC package. The GPS module 1227 may communicate via network 1162 with the EHR server 1106, or some other location data service to determine location information, for example GPS coordinates.

The RF module 1229 may transmit/receive, for example, a communication signal (e.g., a Radio Frequency (RF) signal). The AR device 140 may transmit and receive data from the mobile device via the RF module 1229. Likewise, the AR device 140 may transmit and receive data from the EHR server 1106 via the RF module 1229. The RF module may transmit a request for location information to the EHR server 1106. The RF module 1229 may include, for example, a transceiver, a Power Amp Module (PAM), a frequency filter, a Low Noise Amplifier (LNA), an antenna, or the like. According to another exemplary embodiment, at least one of the cellular module 1221, the WiFi module 1223, the BT module 1225, the GPS module 1227, and the NFC module 1228 may transmit/receive an RF signal via a separate RF module.

The subscriber identity module 1224 may include, for example, a card including the subscriber identity module and/or an embedded SIM, and may include unique identification information (e.g., an Integrated Circuit Card IDentifier (ICCID)) or subscriber information (e.g., an International Mobile Subscriber Identity (IMSI)).

The memory 1230 (e.g., the memory 1130) may include, for example, an internal memory 1232 or an external memory 1234. The internal memory 1232 may include, for example, at least one of a volatile memory (e.g., a Dynamic RAM (DRAM), a Static RAM (SRAM), a Synchronous Dynamic RAM (SDRAM), etc.) and a non-volatile memory (e.g., a One Time Programmable ROM (OTPROM), a Programmable ROM (PROM), an Erasable and Programmable ROM (EPROM), an Electrically Erasable and Programmable ROM (EEPROM), a mask ROM, a flash ROM, a flash memory (e.g., a NAND flash memory, a NOR flash memory, etc.), a hard drive, or a Solid State Drive (SSD)).

The external memory 1234 may further include a flash drive, for example, Compact Flash (CF), Secure Digital (SD), Micro Secure Digital (Micro-SD), Mini Secure digital (Mini-SD), extreme Digital (xD), memory stick, or the like. The external memory 1234 may be operatively and/or physically connected to the AR device 140 via various interfaces.

The sensor module 1240 may measure, for example, physical quantity or detect an operational status of the AR device 140, and may convert the measured or detected information into an electric signal. The sensor module 1240 may include, for example, at least one of a gesture sensor 1240A, a gyro sensor 1240B, a pressure sensor 1240C, a magnetic sensor 1240D, an acceleration sensor 1240E, a grip sensor 1240F, a proximity sensor 1240G, a color sensor 1240H (e.g., a Red, Green, Blue (RGB) sensor), a bio sensor 1240I, a temperature/humidity sensor 1240J, an illumination sensor 1240K, an Ultra Violet (UV) sensor 1240M, an ultrasonic sensor 1240N, and an optical sensor 1240P. Proximity sensor 1240G may comprise LIDAR, radar, sonar, time-of-flight, infrared or other proximity sensing technologies. The gesture sensor 1240A may determine a gesture associated with the AR device 140. For example, as the AR device 140 moves the AR device 140 may move in a particular way so as to execute, for example, a game action. The gyro sensor 1240B may be configured to determine a manipulation of the AR device 140 in space, for example if the AR device 140 is located on a user's head, the gyro sensor 1240B may determine the user has rotated the user's head a certain number of degrees. Accordingly, the gyro sensor 1240B may communicate a degree of rotation to the processor 1212 so as to adjust the augmented reality scene 123 by the certain number of degrees and accordingly maintaining the position of the virtual object as rendered within the augmented reality scene. The proximity sensor 1240G may be configured to use sonar, radar, LIDAR, or any other suitable means to determine a proximity between the AR device and the one or more physical objects. For instance, the proximity sensor 1240G may determine the proximity of one or more physical objects, including the image cube 150. The proximity sensor 1240G may communicate the one or more physical objects such ast the image cube 150 to the processor 1212 so the virtual object may be correctly rendered. The ultrasonic sensor 1240N may also be likewise configured to employ sonar, radar, LIDAR, and the like to determine the ultrasonic sensor may emit and receive acoustic signals and convert the acoustic signals into electrical signal data. The electrical signal data may be communicated to the processor 1212 and used to determine any of the image data, spatial data, or the like. According to one exemplary embodiment, the optical sensor 1240P may detect ambient light and/or light reflected by an external object (e.g., a user's finger. etc.), and which is converted into a specific wavelength band by means of a light converting member. Additionally or alternatively, the sensor module 1240 may include, for example, an E-nose sensor, an ElectroMyoGraphy (EMG) sensor, an ElectroEncephaloGram (EEG) sensor, an ElectroCardioGram (ECG) sensor, a Magenetic Resonance Imaging (MRI) sensor, an Infrared (IR) sensor, an iris sensor, and/or a fingerprint sensor. The sensor module 1240 may further include a control circuit for controlling at least one or more sensors included therein. In a certain exemplary embodiment, the AR device 140 may further include a processor configured to control the sensor module 1204 either separately or as one part of the processor 1212, and may control the sensor module 1240 while the processor 1212 is in a sleep state.

The input device 1250 may include, for example, a touch panel 1252, a (digital) pen sensor 1254, a key 1256, or an ultrasonic input device 1258. The touch panel 1252 may recognize a touch input, for example, by using at least one of an electrostatic type, a pressure-sensitive type, and an ultrasonic type. In addition, the touch panel 1252 may further include a control circuit. The touch panel 1252 may further include a tactile layer and thus may provide the user with a tactile reaction.

The (digital) pen sensor 1254 may be, for example, one part of a touch panel, or may include an additional sheet for recognition. The key 1256 may be, for example, a physical button, an optical key, a keypad, or a touch key. The ultrasonic input device 1258 may detect an ultrasonic wave generated from an input means through a microphone (e.g., a microphone 1288) to confirm data corresponding to the detected ultrasonic wave.

The display 1260 (e.g., the display 1260) may include a panel 1262, a hologram unit 1264, or a projector 1266. The panel 1262 may be implemented, for example, in a flexible, transparent, or wearable manner. The panel 1262 may be constructed as one module with the touch panel 1252. According to one exemplary embodiment, the panel 1262 may include a pressure sensor (or a force sensor) capable of measuring strength of pressure for a user's touch. The pressure sensor may be implemented in an integral form with respect to the touch panel 1252, or may be implemented as one or more sensors separated from the touch panel 1252.

The hologram unit 1264 may use an interference of light and show a stereoscopic image in the air. The projector 1266 may display an image by projecting a light beam onto a screen. The screen may be located, for example, inside or outside the AR device 140. According to one exemplary embodiment, the display 1260 may further include a control circuit for controlling the panel 1262, the hologram unit 1264, or the projector 1266.

The display 1060 may the augmented reality scene. The display 1260 may receive image data captured by camera module 1291 from the processor 1212. The display 1260 may display the image data. The display 1260 may display the one or more physical objects. The display 1260 may display one or more virtual objects.

The interface 1270 may include, for example, a High-Definition Multimedia Interface (HDMI) 1272, a Universal Serial Bus (USB) 1274, an optical communication interface 1276, or a D-subminiature (D-sub) 1278. The interface 1270 may be included, for example, in the communication interface 1170 of FIG. 1. Additionally or alternatively, the interface 1270 may include, for example, a Mobile High-definition Link (MHL) interface, a Secure Digital (SD)/Multi-Media Card (MMC) interface, or an Infrared Data Association (IrDA) standard interface.

The audio module 1280 may bilaterally convert, for example, a sound and electric signal. At least some constitutional elements of the audio module 1280 may be included in, for example, the input/output interface 1150 of FIG. 11. The audio module 1280 may convert sound information which is input or output, for example, through a speaker 1282, a receiver 1284, an earphone 1286, the microphone 1288, or the like.

The camera module 1291 is, for example, a device for image and video capturing, and according to one exemplary embodiment, may include one or more image sensors (e.g., a front sensor or a rear sensor), a lens, an Image Signal Processor (ISP), or a flash (e.g., LED or xenon lamp). The camera module 1291 may comprise a forward facing camera for capturing a scene. The camera module 1291 may also comprise a rear-facing camera for capturing eye-movements or changes in gaze.

The power management module 1295 may manage, for example, power of the AR device 140. According to one exemplary embodiment, the power management module 1295 may include a Power Management Integrated Circuit (PMIC), a charger Integrated Circuit (IC), or a battery fuel gauge. The PMIC may have a wired and/or wireless charging type. The wireless charging type may include, for example, a magnetic resonance type, a magnetic induction type, an electromagnetic type, or the like, and may further include an additional circuit for wireless charging, for example, a coil loop, a resonant circuit, a rectifier, or the like. The battery gauge may measure, for example, residual quantity of the battery 1296 and voltage, current, and temperature during charging. The battery 1296 may include, for example, a rechargeable battery and/or a solar battery.

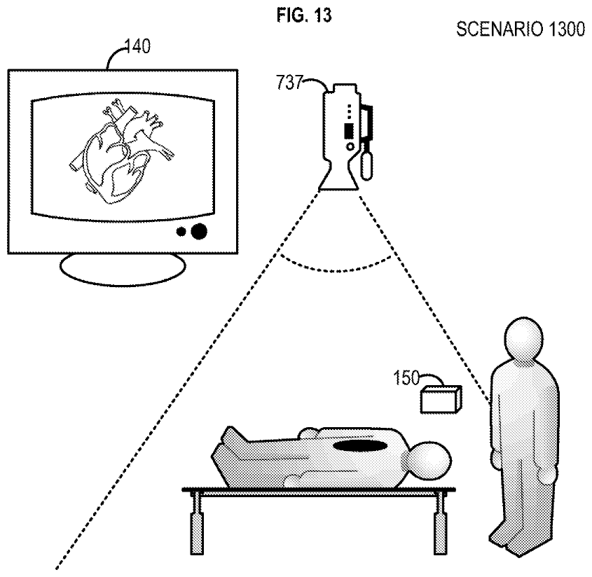

The indicator 1297 may display a specific state, for example, a booting state, a message state, a charging state, or the like, of the AR device 140 or one part thereof (e.g., the processor 1212). The motor 1298 may convert an electric signal into a mechanical vibration, and may generate a vibration or haptic effect. Although not shown, the AR device 140 may include a processing device (e.g., a GPU) for supporting a mobile TV. The processing device for supporting the mobile TV may process media data conforming to a protocol of, for example, Digital Multimedia Broadcasting (DMB), Digital Video Broadcasting (DVB), MediaFlo™, or the like. FIG. 13 is an example implementation scenario 1301 of the disclosed methods, systems, and apparatus. It shows an operating room comprising the image cube 150, the camera 737, and the AR device 140.

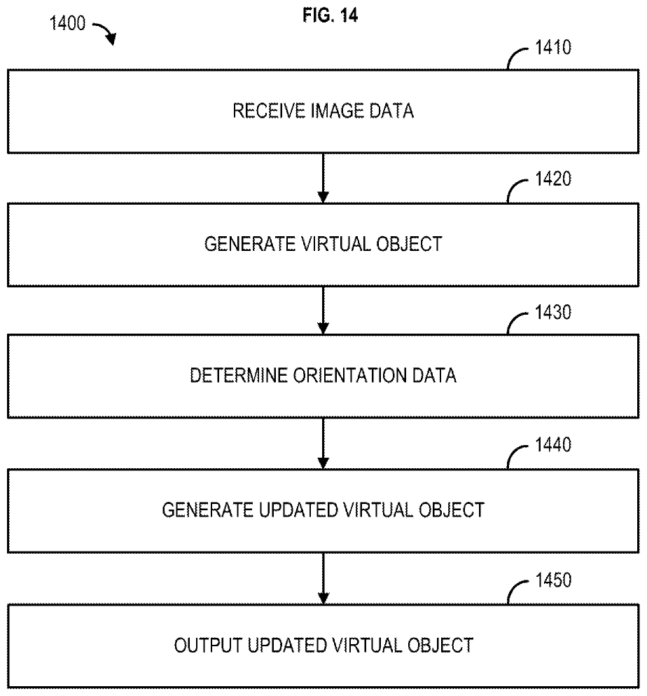

FIG. 14 is an example method 1400 that may be implemented in whole or in part, by one or more of the AR device 140 or any other suitable computing device as disclosed herein.

At step 1410 image data is determined. Examples of imaging data sets to be used include, but are not limited to, medical imaging datasets such as CT, MRI, ultrasound and fluoroscopic images, reference atlas data sets of anatomy, surgical procedures or surgical instruments, photographs, sketches or renderings, as well as animations. The specific image data within a set of possibilities may be determined by a camera, specified by where the cube is with respect to the patient or the user (for example, if the cube is held over the patient's head, images of the brain are brought up, and if the cube is moved down towards the patient's torso, the ribcage is rendered instead). The image data may be captured by an image capture device (e.g., the camera 151), specified by a user (e.g., uploaded by a proceduralist), or determined by any other suitable means. In an aspect, the image data may comprise at least one virtual representation of a real world scene within the field of view of the camera 151. In an aspect, the image data may comprise at least one virtual representation of a real world scene with a field of view of the AR device 140. In an aspect the image data may be captured by the camera module 1091 or other sensors such as the proximity sensor 1040G, biometric sensor 10401, illumination sensor 1040K, or UV sensor 1040M. In an aspect, the proximity sensor 1040G may employ LIDAR, radar, sonar, or the like or may determine the proximity of one or more physical objects by determining whether the object is in focus according to an aperture setting. The proximity sensor 1040G may determine the presence of one or more physical objects within the field of view 900 as well as the distance between the AR device 140 and one or more physical objects. The camera module 1091 may capture video data or still-image data. In an aspect, the field of view 900 of the AR device 140 may be determined based on the image data, for instance by overlaying a grid comprising the 3D spatial coordinate system 902 over the image. For example, the image data may comprise parameters such as width, height, distance, volume, shape, etc. In an aspect, the field of view may comprise a virtual object and/or the one or more physical objects. In an aspect the virtual representation of the one or more physical objects may comprise a likeness of the one or more physical objects output on a display, for example by the AR device 140. The camera may comprise a still-frame camera, a video camera, or any suitable image capture technology as described herein. The image data may also be received from a server, or instance the EHR server. The image data may be associated with a physical object, such as a human heart or some other organ. Likewise, the image data may be associated with a physical object such as a surgical implement. In an aspect, the image data may be retrieved from a database. For example, the image data may be retrieved from the EHR server. The image data received from the EHR server may be associated with a patient. For example, the image data received from the EHR server may be associated with a patient identifier such as a name or a number. In an aspect, the image data received from the EHR server may be compared to the image data captured by the camera. For example, the computing device may use known object recognition techniques to determine that the patient undergoing the procedure is the same patient associated with the image data retrieved from the EHR server. In an aspect, the proceduralist may confirm that the patient undergoing the procedure is the same patient that is associated with the image data received from the EHR server. For example, upon receiving the image data from the EHR server, the proceduralist may be prompted to confirm the identity of the patient. For example, a prompt may appear on the user interface.

At step 1420, a virtual object may be generated. The virtual object may comprise a virtual representation of a physical object. The virtual object may be generated based on the image data associated with the physical object such as the human heart or surgical implement. The image cube may comprise a material which has sufficient contrast so as to be imaged by a camera mounted above or adjacent to the surgical field and thereby facilitate the rendering of a virtual object. The location of the image cube in space may be determined by a variety of sensors such as computer vision sensors so as to determine an orientation of the image cube and a position in space of the image cube. The image cube may be in communication with a plurality of computing devices including servers, displays, image capture technologies and the like. The system may associate an augmented reality or virtual reality image of, for example, a body part, with the image cube. For example, the orientation of the augmented or virtual reality body part may be synchronized with the orientation of the image cube may anchoring (e.g., virtually registering) features of the augmented or virtual reality heart with the features of the image cube. Thus, a proceduralist may interact with the AR or VR image of the body part by manipulating the image cube.

At step 1430, orientation data may be determined. The orientation data may be determined according to the techniques described herein as they relate to the image cube. For instance, the camera, or any other suitable sensor may determine the orientation of the image cube and associate that orientation with an orientation of the physical object and/or an orientation of the virtual object. For instance, a plurality of planes and/or axis may be determined based on the orientation of the image cube and thereafter associated with the orientation of the physical object and/or the orientation of the virtual object. As such, a change in orientation of the image cube may result in a change in the orientation of the virtual object. The orientation data may include, for example, data associated with roll, pitch, and yaw rotations. Additional sensors and/or data may be obtained, for example, LIDAR, radar, sonar, signal data (e.g., received signal strength data), and the like.

At step 1440, an updated virtual object may be generated. As described above, the updated virtual object may comprise an updated orientation. The updated orientation may be a result of a user input such as a manipulation of the image cube captured by the camera and rendered by the AR device. The spatial data associated with the one or more virtual objects may be registered to spatial data image associated with the image cube data. Registering may refer to determining the position of a given virtual object of the one or more virtual objects w/in the scene. Registering may also refer to the position of the virtual object relative any of the one or more physical objects in the augmented reality scene. The system may associate an augmented reality or virtual reality image of, for example, a body part, with the image cube. Thereby, a proceduralist may interact with the AR or VR image of the body part by manipulating the image cube.

At step 1450, the updated virtual object may output. For example, the AR device may be caused to display the updated virtual object.

For purposes of illustration, application programs and other executable program components are illustrated herein as discrete blocks, although it is recognized that such programs and components can reside at various times in different storage components. An implementation of the described methods can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” can comprise volatile and non-volatile, removable and non-removable media implemented in any methods or technology for storage of information such as computer readable instructions, data structures, program modules, or other data. Exemplary computer storage media can comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A method comprising:
receiving image data associated with a physical object;
generating, based on the image data associated with the physical object, a virtual object;
determining, based on an image cube comprising scannable features, orientation data wherein the orientation data is indicative of an orientation of the virtual object and wherein the orientation of the virtual object is associated with the orientation of the image cube;
generating, based on a received input, an updated virtual object wherein the updated virtual object comprises an updated orientation of the virtual object, and wherein the received input comprises a manipulation of the physical object; and
output the updated virtual object.

2. The method of claim 1, wherein generating the virtual object and generating he updated virtual object comprise synchronizing virtual object orientation data with physical object orientation data.

3. The method of claim 1, wherein outputting the updated virtual object comprises causing an augmented reality device to display the updated virtual object.

4. The method of claim 1, wherein each of the virtual object and the updated virtual object are manipulable.

5. The method of claim 1, wherein the image cube comprises a sterilizable image cube, which can be brought into a sterile procedural field.

6. The method of claim 1, wherein receiving the image data associated with the physical object comprises receiving the image data associated with physical object from a camera.

7. The method of claim 1, wherein the scannable features comprise at least one of a quick response (QR) code or Universal Product Code (UPC).

8. A system comprising:
a first computing device configured to:
receive image data associated with a physical object;
generate, based on the image data associated with the physical object, a virtual object;
determine, based on an image cube comprising scannable features, orientation data wherein the orientation data is indicative of an orientation of the virtual object and wherein the orientation of the virtual object is associated with the orientation of the image cube;
generate, based on a received input, an updated virtual object wherein the updated virtual object comprises an updated orientation of the virtual object, and wherein the received input comprises a manipulation of the physical object; and
a display device configured to:
output the updated virtual object.

9. The system of claim 8, wherein the first computing device is configured to receive the image data associated with the physical object by receiving the image data associated with the physical object from an electronic health record (EHR) database.

10. The system of claim 9, wherein the first computing device is configured to receive the image data associated with the physical object by decrypting an encrypted electronic health record so as to be compliant with the health insurance portability and accountability act (HIPAA).

11. The system of claim 8, wherein the first computing device is configured to receive the image data associated with the physical object by receiving the image data associated with physical object from a camera.

12. The system of claim 8, wherein each of the virtual object and the updated virtual object are manipulable.

13. The system of claim 8, wherein the image cube comprises a sterilizable image cube.

14. The system of claim 8, wherein the scannable features comprise at least one of a quick response (QR) code or Universal Product Code (UPC).

15. An apparatus comprising:
one or more processors; and
a memory storing processor executable instructions that, when executed by the one or more processors, cause the apparatus to:
receive image data associated with a physical object;
generate, based on the image data associated with the physical object, a virtual object;
determine, based on an image cube comprising scannable features, orientation data wherein the orientation data is indicative of an orientation of the virtual object and wherein the orientation of the virtual object is associated with the orientation of the image cube;
generate, based on a received input, an updated virtual object wherein the updated virtual object comprises an updated orientation of the virtual object, and wherein the received input comprises a manipulation of the physical object; and
output the updated virtual object.

16. The apparatus of claim 15, wherein the processor executable instructions that, when executed by the one or more processors, cause the apparatus to receive the image data associated with the physical object, further cause the apparatus to receive the image data associated with the physical object by receiving the image data associated with the physical object from an electronic health record (EHR) database.

17. The apparatus of claim 15, wherein the processor executable instructions that, when executed by the one or more processors, cause the apparatus to receive the image data associated with the physical object, further cause the apparatus to receive the image data associated with the physical object by decrypting an encrypted electronic health record so as to be compliant with the health insurance portability and accountability act (HIPAA).

18. The apparatus of claim 15, wherein each of the virtual object and the updated virtual object are manipulable.

19. The apparatus of claim 15, wherein the image cube comprises a sterilizable image cube.

20. The apparatus of claim 15, wherein the scannable features comprise at least one of a quick response (QR) code or Universal Product Code (UPC).
